(19) United States
(12) Patent Application Publication
(10) Pub. No.: US 2026/0159807 A1
(43) Pub. Date: Jun. 11, 2026

(54) Task-Based Learning in Cortical Organoids

(71) Applicant: The Regents of the University of California, Oakland, CA (US)

(72) Inventors: Mircea Teodorescu, Santa Cruz, CA (US); David Haussler, Santa Cruz, CA (US); Ash Robbins, Santa Cruz, CA (US); Alex Spaeth, Santa Cruz, CA (US)

(73) Assignee: The Regents of The University of California, Oakland, CA (US)

(21) Appl. No.: 19/411,629

(22) Filed: Dec. 8, 2025

Related U.S. Application Data
(60) Provisional application No. 63/729,211, filed on Dec. 6, 2024.

(51) Int. Cl. C12N 5/0793; C12M 1/34; C12M 1/36; C12M 1/42; C12M 3/00; G16B 5/00; G16B 40/20

(52) U.S. Cl. CPC C12N 5/0619; C12M 21/08; C12M 35/02; C12M 41/46; C12M 41/48; G16B 5/00; G16B 40/20

(57) ABSTRACT

The present disclosure relates to systems and methods for inducing adaptive learning in a biological neural network cultured in vitro. A multi-electrode array interfaces with the biological neural network and includes a plurality of recording electrodes and a plurality of stimulation electrodes. One or more processors characterize the neural network by delivering electrical stimulation to a plurality of putative neural units and measuring stimulus-evoked responses and select a neural configuration comprising at least one input neural unit, at least one output neural unit, and a plurality of training neural units. A simulated task is operated in closed loop with the biological neural network by encoding one or more task state variables as electrical stimulation delivered to the input neural unit, decoding control signals from activity of the output neural unit, and updating the simulated task.

### PRIORITY DATA

This application claims the benefit of U.S. Patent Application No. 63/729,211, entitled “Task-Based Learning in Cortical Organoids,” filed on Dec. 6, 2024 (Attorney Docket No. UCSC1002USP01). The provisional patent application is incorporated by reference for all purposes.

### FIELD OF THE TECHNOLOGY DISCLOSED

The present disclosure relates to neural engineering and electrophysiology, and more particularly to systems and methods for interfacing with in vitro biological neural networks, including cortical organoids, for studying information processing and adaptive neural behavior.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Biological neurons are capable of nonlinear and dynamic information processing, surpassing artificial systems that often require multiple computational layers to approximate the functional behavior of a single neuron. Contemporary electrophysiological interfacing techniques enable the controlled encoding of information into neural tissue, the decoding of neuronal activity, and the modulation of network dynamics across distinct plasticity timescales. Such capabilities provide an essential foundation for advancing the scientific understanding of biological learning mechanisms and for enabling future developments in therapeutic neuromodulation and biologically inspired computation.

In vivo learning processes typically rely on reinforcement learning principles and Hebbian plasticity, facilitated by neuromodulatory pathways, including dopaminergic systems. In contrast, in vitro neural preparations lack these multi-regional and modulatory structures, and therefore, translating known biological learning rules into reliable, goal-directed training of isolated neural tissue has remained a longstanding challenge. Nonetheless, establishing robust in vitro learning frameworks remains of substantial interest, given the potential to leverage adaptive biological circuitry and to elucidate mesoscale principles relevant to neuroscience, neurotechnology, and computational modeling.

Traditional dissociated neuronal cultures generally lack the architectural organization characteristic of developing brain tissue. By comparison, brain organoids derived from pluripotent stem cells can recapitulate several structural and functional features of early cortical development, including heterogeneous neuronal populations, layered arrangements, and spontaneous oscillatory activity. Despite these advances, many studies involving organoids and other in vitro systems have focused primarily on spontaneous phenomena such as bursting, functional connectivity, and waveform characteristics, owing in part to the absence of structured external input.

High-density microelectrode arrays offer refined access to neuronal populations by enabling simultaneous electrical stimulation and multisite recording with high spatial and temporal resolution. These platforms support the identification of putative neuronal units, the characterization of spatiotemporal activity patterns, and the systematic assessment of stimulus-evoked responses. Moreover, such arrays are well suited to closed-loop experimental paradigms that seek to relate neural activity to controlled perturbations and feedback processes.

Over several decades, multiple stimulation strategies have been investigated to influence and shape the activity of in vitro neural networks. Early approaches employed low-frequency stimulation to evoke network-level bursting for supervised training, later formalized under the concept of learning by stimulation avoidance. Additional research embodied cultured neural networks into robotic or virtual systems, allowing neural activity to govern externally observable behaviors. High-frequency tetanic stimulation has been widely utilized to induce synaptic plasticity, facilitate pattern recognition tasks, or modify bursting dynamics. More recent work has explored computational frameworks such as reservoir computing and theoretical constructs such as the free energy principle to interpret or exploit neural dynamics, although issues of reproducibility and interpretability remain under discussion.

A further category of techniques employs discrete high-frequency training pulses intended to drive associative plasticity. While tetanic stimulation can reliably induce synaptic modification, practical challenges persist regarding the selection of neuronal targets, the choice of stimulation frequencies and pulse structures, and the timing of stimulation relative to ongoing neural activity. These challenges continue to motivate the development of systematic, closed-loop methodologies for evaluating and understanding adaptive processes within in vitro neural systems.

### SUMMARY

In one embodiment, a system for inducing adaptive learning in a biological neural network cultured in vitro is described, the system comprising a multi-electrode array configured to interface with the biological neural network and comprising a plurality of recording electrodes and a plurality of stimulation electrodes, and one or more processors and a memory storing instructions that, when executed, cause the system to characterize the neural network by delivering electrical stimulation to a plurality of putative neural units and measuring responses. The one or more processors and the memory storing instructions, when executed, further cause the system to select, based on the characterization, a neural configuration comprising at least one input neural unit to receive electrical stimulation encoding task information, at least one output neural unit provide electrical activity for decoding control signals, and a plurality of training neural units to receive training stimulation. The one or more processors and the memory storing instructions, when executed, further cause the system to operate a simulated task in a closed loop with the biological neural network by iteratively encoding one or more task state variables as electrical stimulation received by the at least one input neural unit, recording electrical activity from the at least one output neural unit in response to the encoded stimulation and decoding the electrical activity into control signals, updating the simulated task based on the control signals, and determining task performance. The one or more processors and the memory storing instructions, when executed, further cause the system to adaptively select training electrical stimulation patterns based on the task performance and deliver the selected training electrical stimulation patterns to the plurality of training neural units.

In another embodiment, a method for inducing goal-directed learning in a biological neural network cultured in vitro is described, the method comprising interfacing the biological neural network with a multi-electrode array comprising a plurality of recording electrodes and a plurality of stimulation electrodes, characterizing the biological neural network by delivering electrical stimulation to a plurality of putative neural units via the plurality of stimulation electrodes and measuring stimulus-evoked responses via the plurality of recording electrodes, and selecting, based on the characterization, a neural configuration comprising at least one input neural unit configured to receive electrical stimulation encoding task information, at least one output neural unit configured to provide electrical activity for decoding control signals, and a plurality of training neural units configured to receive training stimulation. The method further comprises operating a simulated task in a closed loop with the biological neural network by iteratively encoding one or more task state variables as electrical stimulation delivered to the at least one input neural unit, recording electrical activity from the at least one output neural unit in response to the encoded stimulation and decoding the recorded electrical activity into control signals, updating the simulated task based on the control signals, and determining task performance. The method further comprises adaptively selecting training electrical stimulation patterns based on the task performance and delivering the selected training electrical stimulation patterns to the plurality of training neural units.

In a further embodiment, a non-transitory computer-readable storage medium storing instructions that, when executed by at least one processor coupled to a multi-electrode array interfacing with a biological neural network cultured in vitro, cause the at least one processor to perform operations is described, the operations comprising interfacing the biological neural network with a multi-electrode array comprising a plurality of recording electrodes and a plurality of stimulation electrodes, characterizing the biological neural network by delivering electrical stimulation to a plurality of putative neural units via the plurality of stimulation electrodes and measuring stimulus-evoked responses via the plurality of recording electrodes, and selecting, based on the characterization, a neural configuration comprising at least one input neural unit configured to receive electrical stimulation encoding task information, at least one output neural unit configured to provide electrical activity for decoding control signals, and a plurality of training neural units configured to receive training stimulation. The operations further comprise operating a simulated task in a closed loop with the biological neural network by iteratively encoding one or more task state variables as electrical stimulation delivered to the at least one input neural unit, recording electrical activity from the at least one output neural unit in response to the encoded stimulation and decoding the recorded electrical activity into control signals, updating the simulated task based on the control signals, and determining task performance. The operations further comprise adaptively selecting training electrical stimulation patterns based on the task performance and delivering the selected training electrical stimulation patterns to the plurality of training neural units.

The foregoing general description of the illustrative embodiments and the following detailed description thereof are merely exemplary aspects of the teachings of this disclosure and are not restrictive.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1A is a schematic representation of a system configured to induce adaptive learning in a biological neural network cultured in vitro.

FIG. 1B is a schematic representation of a multiphase experimental design comprising a record phase, a stimulation phase, and a closed-loop training phase.

FIG. 1C is a flowchart representation of episode-level closed-loop operation between a biological neural network and a simulated dynamical environment.

FIG. 1D is an illustration of a representative stimulation schedule showing temporally separated stimulation epochs delivered to a plurality of stimulation electrodes, according to certain embodiments.

FIG. 1E is a schematic overview of an experimental workflow implemented across a plurality of organoids, illustrating an example sequence of record, stimulation, and training phases, associated training cycles, and episodic closed-loop interactions with a simulated environment, in accordance with some embodiments.

FIG. 2A is a schematic representation of directed patterning and self-organization of embryonic stem cells during cortical organoid generation.

FIG. 2B is a depiction of a three-dimensional cortical organoid exhibiting structural organization and developing neural tissue architecture.

FIG. 2C shows differentiated neuronal subtypes expressing layer-specific and cell-type-specific markers during organoid maturation.

FIG. 2D is a representation of a high-density microelectrode array interfaced with a cortical organoid for stimulation and recording.

FIG. 3A is a depiction of stimulus-evoked neural responses showing short-latency spike activity and corresponding latency histograms.

FIG. 3B is a schematic representation of first-order causal connectivity illustrating the probability of direct stimulus-evoked responses between neural units.

FIG. 3C is a depiction of stimulus-evoked network-mediated bursting responses representing multi-order activity.

FIG. 3D is a representation of multi-order causal connectivity showing mean spike responses within extended post-stimulus time windows.

FIG. 3E is a schematic representation of selected neural roles for encoding and decoding based on first-order causal connectivity.

FIG. 3F is a schematic representation of selected neural roles for encoding and decoding based on multi-order causal connectivity.

FIG. 3G is a heatmap representation illustrating stimulus-evoked spike counts across stimulation electrodes and stimulation repetitions, with burst-classified events indicated for visualization.

FIG. 3H is a raster diagram of burst-evoking stimulation repetitions showing temporally clustered spike activity corresponding to periods of network-wide bursting.

FIG. 3I is a raster diagram of non-burst-evoking stimulation repetitions showing distributed spike activity in the absence of network-wide bursts.

FIG. 4A is a depiction of a biphasic square-wave stimulation pulse and the temporal organization of multi-pulse training patterns.

FIG. 4B is an illustration of three training paradigms comprising a null condition, a random stimulation condition, and an adaptive stimulation condition.

FIG. 4C is a representation of cartpole task performance across sequential training cycles under varying stimulation paradigms.

FIG. 4D is an illustration of mean and interquartile performance values for each stimulation paradigm within a representative trial.

FIG. 4E is a depiction of overlaid pole-angle trajectories across selected training cycles.

FIG. 4F is a representation of episode-level performance and adaptive training-pulse delivery times within individual training cycles.

FIG. 5A is a depiction of long-duration performance in a continuous adaptive training paradigm.

FIG. 5B is a representation of improvement metrics for training-pulse combinations evaluated across training sessions.

FIG. 5C shows temporal progression of task performance together with corresponding value-estimate updates for training stimulation patterns.

FIG. 5D is a depiction of a sigmoid-type estimation of the control policy formed by a biological neural network during a training cycle.

FIG. 5E is a depiction of sigmoid-type control policy estimations shown for selected training epochs.

FIG. 5F is a representation of early-episode input-output flow fields illustrating neural response patterns under initial training conditions.

FIG. 5G is a representation of late-episode input-output flow fields illustrating stabilized neural response patterns near the balancing region.

FIG. 6A is a depiction of performance distributions across null, random, adaptive, and continuous-adaptive stimulation paradigms.

FIG. 6B is an illustration of the fraction of training cycles that achieved proficiency under each stimulation paradigm.

FIG. 6C is a representation of performance predicted by functional connectivity measured during baseline recording.

FIG. 6D is a representation of performance predicted by first-order causal connectivity measured during stimulus-evoked characterization.

FIG. 6E is a depiction of correlations between connectivity features and task performance for input and output neural units.

FIG. 7 is illustrates a computing system for implementing one or more computational aspects of the present disclosure.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Biological neural networks cultured in vitro, including dissociated neuronal cultures and brain organoids derived from pluripotent stem cells, exhibit rich nonlinear and dynamic information-processing capabilities that can surpass those of artificial systems, which often require multiple layers to approximate the behavior of a single biological neuron. Modern electrophysiological interfaces, such as high-density microelectrode arrays, enable experimenters to encode information into neural tissue, decode information from neuronal activity, and perturb underlying network dynamics across various plasticity timescales. In vivo, learning typically arises from reinforcement learning and Hebbian plasticity supported by neuromodulatory systems, including dopaminergic pathways. By contrast, in vitro neural systems lack these multi-regional and modulatory circuits, and existing approaches have not yet produced robust, repeatable methods for training isolated neural tissue in a consistent, goal-directed manner.

Aspects of the technology provide a closed-loop electrophysiology framework that systematically interfaces a biological neural network cultured in vitro with a simulated dynamical task environment. Unlike prior in vitro studies that primarily analyze spontaneous bursting or use ad hoc stimulation schemes, the disclosed framework characterizes causal connectivity between putative neural units using stimulus-evoked responses, selects distinct neural roles for encoding, decoding, and training, and couples the biological neural network to an unstable control task that demands continuous, performance-dependent adaptation. The system employs a multi-electrode array to deliver electrical stimulation and record neuronal activity, assigns input neural units to receive electrical stimulation encoding task information, assigns output neural units to provide electrical activity for decoding control signals, and assigns training neural units to receive training electrical stimulation patterns that are adaptively selected based on measured task performance.

Existing in vitro learning paradigms and embodied neural systems exhibit several limitations. Many rely on low-frequency stimulation that evokes network-wide bursts, high-frequency tetanic stimulation applied without principled selection of stimulation targets, or reservoir-computing schemes that depend heavily on external machine-learning readouts. These approaches often lack a systematic method for identifying which neurons to stimulate, what stimulation parameters to use, and when to administer stimulation relative to ongoing behavior. Moreover, they typically do not distinguish between neurons that encode task state, neurons that generate control outputs, and neurons designated specifically for training. As a result, it remains difficult to determine how network connectivity, stimulation patterns, and task structure jointly contribute to goal-directed learning in vitro, and how to compare biological performance against well-defined benchmarks over longitudinal experiments.

The technology disclosed addresses these deficiencies by introducing a system and method that (i) characterize the biological neural network by delivering electrical stimulation to a plurality of putative neural units and computing connectivity information describing directional influence between the putative neural units; (ii) select, based on this connectivity information, a neural configuration comprising at least one input neural unit configured to receive electrical stimulation encoding task information, at least one output neural unit configured to provide electrical activity for decoding control signals, and a plurality of training neural units configured to receive training stimulation; and (iii) operate a simulated task in a closed loop with the biological neural network by iteratively encoding task state as electrical stimulation delivered to the input neural unit or units, decoding control signals from the output neural unit or units, updating the simulated task based on the decoded control signals, and determining task performance. The system further adaptively selects training electrical stimulation patterns based on the task performance and delivers the selected training patterns to the training neural units, thereby enabling performance-dependent modification of network dynamics.

In some embodiments, the simulated task comprises an unstable dynamical system, such as an inverted-pendulum or cartpole system, that requires continuous active control to maintain a system state within prescribed bounds and yields scalar performance metrics based on episode duration or stability. By embodying the biological neural network into such a dynamical task and by using connectivity-informed selection of input, output, and training neural units together with adaptive training stimulation patterns, the disclosed framework provides a principled means to induce and evaluate goal-directed learning in vitro. This architecture transforms in vitro neural networks from passively observed systems into actively trained controllers operating within a standardized, quantitatively defined task environment.

A system and method are therefore provided for inducing adaptive learning in a biological neural network cultured in vitro by interfacing the biological neural network with a multi-electrode array, characterizing connectivity among putative neural units using stimulus-evoked responses, selecting distinct input, output, and training neural units based on the characterization, and operating a simulated task in a closed loop with the biological neural network. During closed-loop operation, one or more processors encode task state as electrical stimulation delivered to the input neural unit or units, decode control signals from electrical activity of the output neural unit or units, update the simulated task based on the decoded control signals, determine task performance, adaptively select training electrical stimulation patterns based on the determined task performance, and deliver the selected training electrical stimulation patterns to the training neural units. In this manner, the technology disclosed enables structured, performance-driven training of in vitro neural systems and establishes a reproducible framework for assessing their learning capabilities.

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

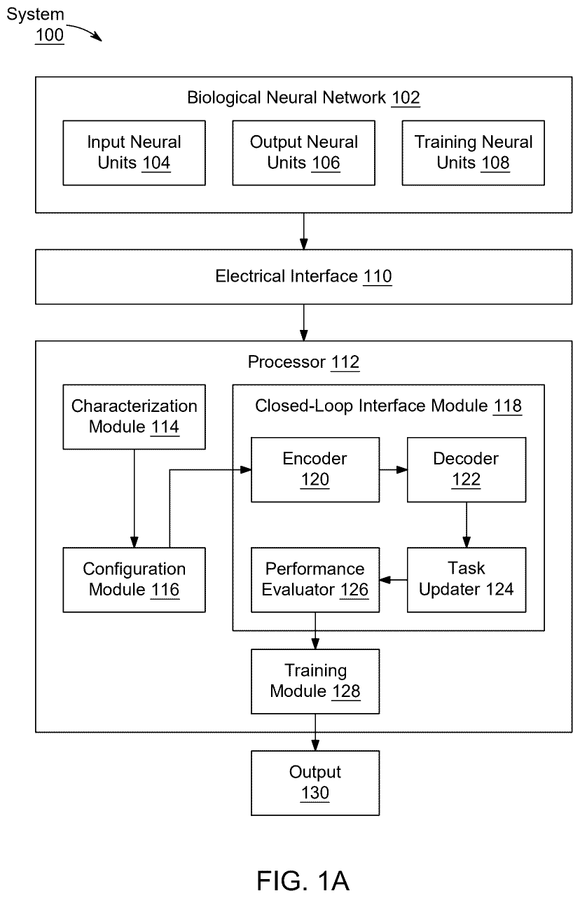

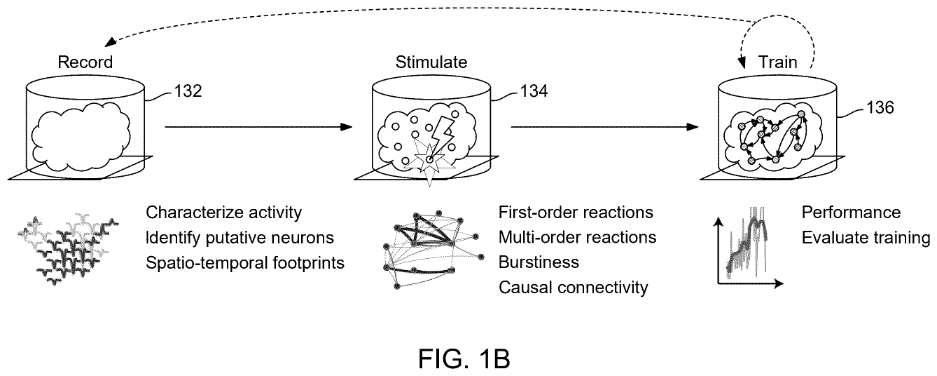

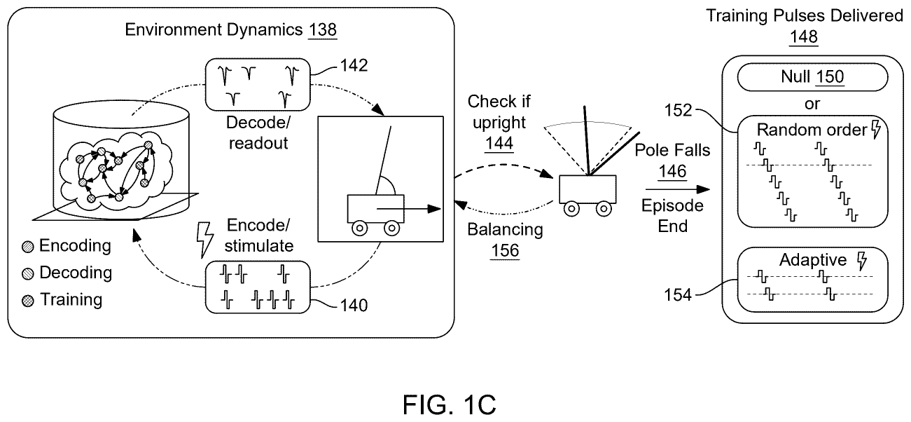

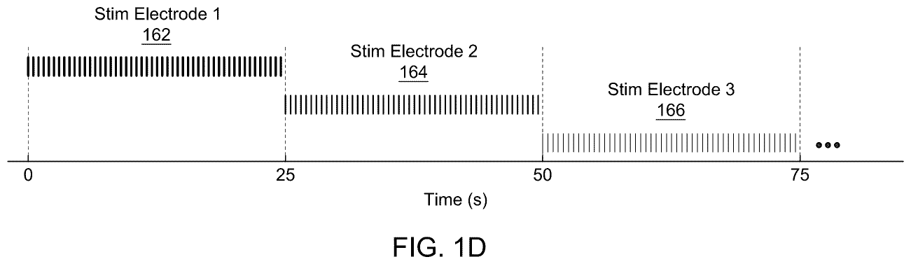

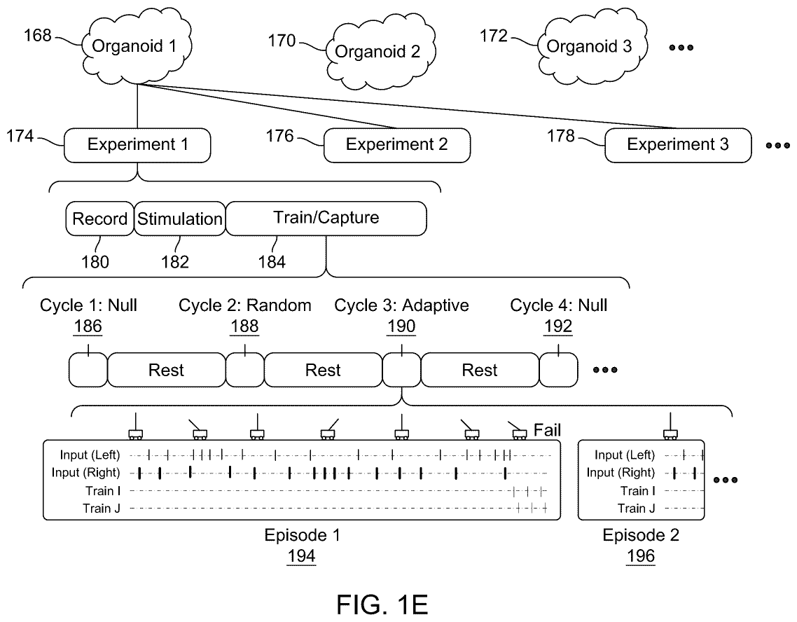

FIG. 1A illustrates a system 100 for inducing adaptive learning in a biological neural network cultured in vitro. The system 100 is configured to establish a closed-loop interface between biological neural tissue and a simulated task environment, such that the biological neural network learns to perform goal-directed control through adaptive training stimulation. The system 100 enables an in vitro neural network to exhibit learning behavior by delivering electrical stimulation patterns that encode task information to designated input neural units, decoding control signals from designated output neural units, and adaptively applying training stimulation to distinct training neural units based on task performance.

### System Architecture

The system 100 comprises a biological neural network 102 cultured in vitro and maintained under controlled physiological conditions. In various embodiments, the biological neural network 102 comprises a cortical organoid derived from pluripotent stem cells cultured for a maturation period sufficient to develop functional neural connectivity and spontaneous electrical activity. The biological neural network 102 may include diverse neuronal subtypes and support rich, spontaneous spiking dynamics suitable for closed-loop electrophysiology.

Within the biological neural network 102, the system 100 designates three functionally distinct populations: input neural units 104, output neural units 106, and training neural units 108. These three populations are mutually exclusive, such that no individual neural unit simultaneously serves more than one role. The input neural units 104 receive electrical stimulation that encodes task-relevant state information from the simulated task environment. The output neural units 106 generate electrical activity that the system 100 records and decodes to produce control signals for the simulated task. The training neural units 108 receive training electrical stimulation patterns that the system 100 adaptively selects based on task performance over time. In certain embodiments, the plurality of training neural units 108 comprises between 8 and 15 neural units that are distinct from both the input neural units 104 and the output neural units 106.

The system 100 further comprises an electrical interface 110 configured to provide bidirectional communication with the biological neural network 102 through electrical stimulation delivery and electrical activity recording. The electrical interface 110 includes a multi-electrode array having a plurality of recording electrodes and a plurality of stimulation electrodes. In some embodiments, the electrical interface 110 comprises a high-density microelectrode array configured to record from and stimulate neural units at the surface of the cortical organoid. The electrodes deliver charge-balanced biphasic electrical pulses and record extracellular voltage fluctuations at sampling rates sufficient to resolve individual action potentials and stimulus-locked responses. The electrical interface 110 thus enables the system 100 to address individual putative neural units and to map their causal interactions.

The system 100 further comprises a processor 112 operably connected to the electrical interface 110. The processor 112 executes computational operations that enable the system 100 to characterize the biological neural network 102, configure neural roles, operate a simulated task in closed loop with the network, evaluate task performance, and adaptively deliver training stimulation. The processor 112 may include one or more processing cores, memory, and associated circuitry configured to implement the functional modules described below. The processor 112 operates with timing precision sufficient to maintain stable real-time closed-loop interaction between the simulated task environment and the biological neural network 102.

### Functional Modules

The processor 112 executes a characterization module 114 configured to characterize connectivity and response properties of the biological neural network 102. The characterization module 114 records spontaneous activity from the electrodes of the electrical interface 110 over a characterization interval to identify a plurality of putative neural units based on their spatiotemporal spike footprints. The characterization module 114 then delivers electrical stimulation pulses to individual putative neural units via selected stimulation electrodes and measures stimulus-evoked responses at the recording electrodes. From these responses, the characterization module 114 computes connectivity information describing directional influence between pairs of putative neural units, including first-order and multi-order causal connectivity metrics. First-order causal connectivity values represent probabilities of direct stimulus-evoked action potentials occurring within a first post-stimulus time window, such as about 10 milliseconds following stimulation. Multi-order causal connectivity values represent network-mediated responses occurring within a longer second post-stimulus time window, such as between about 10 milliseconds and about 200 milliseconds following stimulation.

A configuration module 116 executes on the processor 112 to select a neural configuration for closed-loop operation. The configuration module 116 receives the connectivity information computed by the characterization module 114 and designates which putative neural units will serve as input neural units 104, output neural units 106, and training neural units 108. The configuration module 116 may select input neural units 104 that exhibit relatively sparse downstream connectivity or low propensity to trigger network-wide bursts, thereby supporting stable encoding of task information. The configuration module 116 may select output neural units 106 based on strong first-order causal connectivity from candidate input neural units 104, for example, by requiring that a first-order causal connectivity value between an input-output pair exceed a threshold probability. The configuration module 116 selects the training neural units 108 to be distinct from the input neural units 104 and the output neural units 106, with the number of training neural units chosen according to design criteria and summarized in one or more configuration tables.

A closed-loop interface module 118 executes on the processor 112 to operate a simulated task in closed loop with the biological neural network 102. The closed-loop interface module 118 comprises an encoder 120, a decoder 122, a task updater 124, and a performance evaluator 126.

The encoder 120 converts task state information from the simulated task into electrical stimulation patterns delivered to the input neural units 104. In some embodiments, the encoder 120 implements a nonlinear rate-coding scheme that maps one or more continuous task-state variables to stimulation frequencies for the input neural units 104. For example, for a task that includes an angular state variable θ, the encoder 120 may compute stimulation frequencies f₁ and f₂ for two input neural units according to:

$${f}_{1} = a \cdot {( - sin ( θ ) + b )}^{n}$$

$${f}_{2} = a \cdot {( sin ( θ ) + b )}^{n}$$

where θ denotes a pole angle in a cartpole task, α represents a scaling constant, b represents an offset constant, and n denotes a nonlinear exponent. In a particular embodiment, a=7, b=0.15, and n=2. The encoder 120 then delivers electrical stimulation to the input neural units 104 at frequencies f₁ and f₂ using the electrical interface 110.

The decoder 122 converts electrical activity recorded from the output neural units 106 into control signals for the simulated task. The decoder 122 detects action potentials from the recorded signals and computes spike counts over defined decoding windows. To obtain a smooth estimate of firing rate over time, the decoder 122 applies exponential smoothing according to:

$${r}_{t} = α \cdot {r}_{t - 1} + ( 1 - α ) \cdot {c}_{t}$$

where rₜ represents the smoothed firing rate at time t, rₜ_{^{−1}} represents the smoothed firing rate at the previous time step, cₜ represents the spike count in the current time window, and α denotes a smoothing parameter between 0 and 1. In some embodiments, α is set to about 0.2. For configurations with two output neural units 106, the decoder 122 generates a control signal, such as a horizontal force applied to a simulated cart, based on a difference between the smoothed firing rates of the two output neural units.

The task updater 124 maintains and updates a simulated task environment that operates as an unstable dynamical system requiring continuous active control to maintain stability. In preferred embodiments, the simulated task comprises an inverted pendulum or cartpole system having a cart movable along a horizontal axis and a pole rotatably attached to the cart. State variables include at least a pole angle θ and a pole angular velocity, and the task updater 124 advances the state of the system according to the applied control signals and the system dynamics. The task updater 124 determines episode termination when the pole angle exceeds a threshold angle from vertical, such as ±16 degrees. Simulation parameters, episode structures, and performance thresholds may be summarized in one or more experimental tables.

The performance evaluator 126 computes task performance metrics based on the simulated task trajectories generated by the task updater 124. In some embodiments, the performance evaluator 126 defines a performance metric as episode duration, measured as the time for which the system remains within specified bounds before failure. The performance evaluator 126 may also aggregate performance across multiple episodes to derive short-term and long-term performance statistics, which the system 100 uses to adapt training stimulation.

The processor 112 further executes a training module 128 configured to adaptively select and deliver training electrical stimulation patterns to the training neural units 108 based on the task performance determined by the performance evaluator 126. The training module 128 maintains value estimates for candidate training stimulation patterns and updates these value estimates using a reinforcement-learning algorithm, such as temporal-difference learning with eligibility traces. In some embodiments, the training module 128 updates a value estimate Vᵢ,t for a candidate training stimulation pattern i at time t according to:

$${V}_{i} {,}_{t + 1} = {V}_{i} {,}_{t} + α ( {R}_{t} - {V}_{i} {,}_{t} ) {E}_{i} {,}_{t}$$

where V_{i,t} denotes the current value estimate, α denotes a learning rate, Rₜ denotes a reward signal derived from task performance, and E_{i,t} denotes an eligibility trace associated with pattern i. The eligibility trace E_{i,t} is updated according to:

$${E}_{i , t} = γ {E}_{i , t - 1} + {I}_{i , t}$$

where γ denotes a decay factor and I_{i,t} indicates whether the candidate training stimulation pattern i was delivered at time t.

Training stimulation patterns generated by the training module 128 comprise sequences of multiple biphasic electrical pulses delivered to one or more of the training neural units 108. In some embodiments, the training module 128 employs patterns with inter-pulse intervals of about 5 milliseconds, repeated at a repetition frequency of about 10 Hz for a duration of about 300 milliseconds. The training module 128 may apply conditional delivery rules, such as delivering a training stimulation pattern only when a short-term performance metric calculated over a recent subset of episodes falls below a long-term performance metric computed over a larger window of episodes.

### System Output and Operation

The system 100 generates an output 130 that reflects the performance of the biological neural network 102 in controlling the simulated task over time. The output 130 may comprise one or more performance metrics, such as proficiency rates or episode durations, as well as summaries comparing adaptive training stimulation to baseline or random-stimulation conditions. In some experiments, the system 100 demonstrates that adaptive selection of training stimulation patterns yields a substantially higher fraction of episodes exceeding a predefined proficiency threshold than either random selection of training patterns or operation without training stimulation. Such results, which may be summarized in comparative tables, indicate that the biological neural network 102 has undergone adaptive learning driven by the training stimulation and closed-loop interaction with the simulated task.

During operation, the system 100 typically proceeds through successive phases. In a characterization phase, the characterization module 114 records spontaneous activity and computes connectivity information among putative neural units. In a configuration phase, the configuration module 116 selects input neural units 104, output neural units 106, and training neural units 108 based on the connectivity information and selection criteria. In a closed-loop training phase, the encoder 120 delivers task-encoding stimulation to the input neural units 104, the decoder 122 generates control signals from the output neural units 106, the task updater 124 advances the simulated task, the performance evaluator 126 computes task performance metrics, and the training module 128 adaptively delivers training stimulation to the training neural units 108. Over multiple episodes and training cycles, the system 100 evaluates whether the biological neural network 102 exhibits improved task performance, thereby confirming that the system 100 induces adaptive learning in the in vitro neural network.

### Multiphase Experiment Environment

FIG. 1B is a schematic diagram illustrating a multiphase experimental design implemented by the system 100. The multiphase experimental design comprises three key phases: a record phase 132, a stimulate phase 134, and a train phase 136. The three phases collectively implement a framework for real-time neural interfacing and evaluation of goal-directed learning in cortical organoids embodied in a dynamical control task. The framework consists of network characterization through spontaneous recording, stimulus-response mapping through targeted electrical stimulation, and closed-loop training in a dynamical task. Each phase builds upon automated analysis from the previous phase to systematically identify and interface with relevant neural circuits and to characterize causal connectivity before attempting to dynamically modify stimulus-evoked responses through training. The framework provides millisecond-precision control to minimize latency between the neural culture and the virtual environment and supports reproducibility through an automated analysis pipeline.

In the record phase 132, indicated by the “Record” label, the system 100 performs spontaneous recording to locate and characterize putative neural units within the biological neural network, which may be a mouse cortical organoid generated from pluripotent stem cells and used as a biological substrate for learning. Record uses a spontaneous recording to locate and characterize putative neural units. The electrical interface 110 and the processor 112 cooperate to record spontaneous electrical activity with millisecond resolution from a plurality of electrodes and to extract spatio-temporal footprints corresponding to individual putative neurons. During this phase, the system characterizes activity, identifies putative neurons, and determines spatio-temporal footprints by detecting action potentials and clustering them into units based on waveform shape and spatial distribution. The resulting set of putative neural units and their footprints defines the substrate for subsequent stimulation and causal analysis.

In the stimulate phase 134, indicated by the “Stimulate” label, the system 100 uses electrical stimulation on each of these units to measure stimulus-evoked activity through different temporal ranges. The electrical interface 110 delivers targeted, charge-balanced stimulation pulses to individual putative neural units, and the recording electrodes simultaneously monitor network-wide responses. The processor 112 analyzes the stimulus-evoked activity to quantify first-order reactions, multi-order reactions, burstiness, and causal connectivity. First-order reactions correspond to short-latency, stimulus-locked responses that occur within a first post-stimulus time window and are used to calculate first-order causal connectivity values representing probabilities of direct stimulus-evoked action potentials. Multi-order reactions correspond to longer-latency, network-mediated responses within a second post-stimulus time window that is longer than the first time window and are used to calculate multi-order causal connectivity values representing network-mediated responses. Burstiness metrics quantify the propensity for network-wide bursts following stimulation. Human experimenters then select putative neuron roles from the causal connectivity analysis, or an automated algorithm implements the same selection criteria, to define a neural configuration comprising at least one input neural unit configured to receive electrical stimulation encoding task information, at least one output neural unit configured to provide electrical activity for decoding control signals, and a plurality of training neural units configured to receive training stimulation. These connectivity matrices, first-order and multi-order causal connectivity values, burstiness measures, and selection outcomes may be summarized in one or more tables.

In the train phase 136, indicated by the “Train” label, the system 100 performs closed-loop training in the dynamical task using the configured input, output, and training neural units. Train consists of repeated interactions with the simulated dynamical environment, organized into episodes. During each episode, the system operates a simulated task in a closed loop with the biological neural network by iteratively encoding one or more task state variables as electrical stimulation delivered to the at least one input neural unit, recording electrical activity from the at least one output neural unit in response to the encoded stimulation and decoding the recorded electrical activity into control signals, updating the simulated task based on the control signals, and determining task performance. The system adaptively selects training electrical stimulation patterns based on the task performance and delivers the selected training electrical stimulation patterns to the plurality of training neural units. Performance traces and training evaluations associated with the train phase 136 are used to evaluate training and to determine whether the cortical organoid has achieved goal-directed learning in the dynamical environment.

### Training Loop

FIG. 1C is a flowchart illustrating each episode of a training loop. The left-hand portion of FIG. 1C depicts environment dynamics 138, which represent the closed-loop interaction between the configured biological neural network and a simulated dynamical task environment. The environment dynamics 138 embody the hypothesis that cortical organoids can achieve goal-directed learning in a dynamical environment and therefore are evaluated on the inverted pendulum control problem commonly known as “cartpole.” This task requires continuous, active stabilization of an inherently unstable system, making it ideal for assessing learning of a fundamental control policy. Its well-studied dynamics provide clear performance metrics to evaluate learning capabilities.

Within the environment dynamics 138, the cortical organoid and the simulated environment interact through encoding, decoding, and training functions, as indicated by the icons for encoding, decoding, and training next to the organoid. An encode/stimulate block 140 represents the encoding of task state information as electrical stimulation delivered to the at least one input neural unit. The encoder converts state variables of the cartpole system, such as a pole angle and a pole angular velocity, into stimulation parameters for the input neural units. In some embodiments, the encoder uses a nonlinear rate-coding scheme in which stimulation frequencies f₁ and f₂ for two input neural units are computed as

$${f}_{1} = a \cdot {( - sin ( θ ) + b )}^{n}$$

$${f}_{2} = a \cdot {( sin ( θ ) + b )}^{n}$$

where θ is the pole angle, a is a scaling constant, b is an offset constant, and n is an exponent, and the resulting stimulation frequencies are implemented as trains of biphasic electrical pulses.

A decode/readout block 142 represents the decoding of electrical activity recorded from the at least one output neural unit into control signals. The decoder detects action potentials from the output neural units, computes spike counts over successive time windows, and applies exponential smoothing to obtain smoothed firing rates according to

$${r}_{t} = α {r}_{t - 1} + ( 1 - α ) {c}_{t} ,$$

where rₜ denotes a smoothed firing rate at time t, rₜ_{^{−1}} denotes a smoothed firing rate at a previous time step, cₜ denotes a spike count in the current time window, and α denotes a smoothing parameter. The decoder generates control signals, such as horizontal forces applied to the cart, based on the smoothed firing rates, for example by computing a difference between smoothed firing rates of two output neural units.

A balancing process 156 links the decoded control signals to the inverted pendulum dynamics. The cartpole system evolves under the applied control signals, and its state is updated. A check-if-upright block 144 continuously monitors whether the pole is held upright within a permissible angular range from vertical. As long as the pole is held upright, the organoid and simulated environment remain in closed-loop interaction, and the episode continues with repeated cycles of encoding, decoding, and environment update. An “episode end” condition is reached at a pole-falls block 146 when the pole falls into an unrecoverable position, for example when the pole angle exceeds a predetermined threshold such as ±16 degrees from vertical. The episode is terminated when the pole falls into an unrecoverable position, and the system records episode duration or other task-performance metrics, which may be summarized in performance tables.

Following termination of the episode, the right-hand portion of FIG. 1C illustrates a training pulses delivered block 148, which governs whether and how training pulses are applied at the end of the episode. Finally, depending on the training paradigm, a training pulse may or may not be delivered. In a null condition 150, no training stimulation is delivered to the training neural units, providing a control condition. In a random-order condition 152, training pulses are delivered in a random order to the training neural units, independent of the measured task performance. In an adaptive condition 154, training pulses are selected adaptively based on task performance. In this adaptive paradigm, the system adaptively selects training electrical stimulation patterns based on the task performance and delivers the selected training electrical stimulation patterns to the plurality of training neural units. The training module maintains value estimates for candidate training stimulation patterns, updates the value estimates based on changes in the task performance, and selects subsequent training stimulation patterns according to the updated value estimates. In some embodiments, the value estimates Vᵢ,t for a candidate training stimulation pattern i are updated according to

$${V}_{i , t + 1} = {V}_{i , t} + α ( {R}_{t} - {V}_{i , t} ) {E}_{i , t} ,$$

where V_{i,t} represents a value estimate at time t, α represents a learning rate, Rₜ represents a reward signal based on task performance such as episode duration, and E_{i,t} represents an eligibility trace for pattern i. The eligibility trace is updated according to

$${E}_{i , t} = γ {E}_{i , t - 1} + {I}_{i , t} ,$$

where γ represents a decay factor and I_{i,t} indicates whether the candidate training stimulation pattern i was delivered. Each training electrical stimulation pattern may comprise a sequence of multiple biphasic electrical pulses delivered to one or more of the training neural units with an inter-pulse interval of about 5 milliseconds and repeated at a repetition frequency of about 10 Hz for a duration of about 300 milliseconds.

Across many episodes, FIG. 1C thus depicts a training loop in which environment dynamics 138, the check-if-upright block 144, the pole-falls condition block 146, and the training pulses delivered block 148 together implement a process whereby the system operates a simulated task in a closed loop with the biological neural network, determines task performance, adaptively selects training electrical stimulation patterns based on the task performance, and delivers the selected training electrical stimulation patterns to the plurality of training neural units. Performance evaluation traces in the train phase 136 of FIG. 1B and the episode outcomes in FIG. 1C are used to evaluate training and to determine whether cortical organoids achieve goal-directed learning in the dynamical environment.

In some embodiments, the closed-loop operation illustrated in FIGS. 1A-1C is implemented in a dynamical control task environment that requires continuous, active stabilization of an unstable system. The simulated task may comprise an inverted pendulum or cartpole system in which a pole is pivotally mounted on a cart that moves along a horizontal axis, and the goal of the control policy is to maintain the pole within defined angular bounds relative to vertical over the course of an episode. Because small deviations in pole angle grow rapidly if left uncorrected, such unstable dynamical systems provide a stringent benchmark for assessing whether the biological neural network 102 has acquired a goal-directed control strategy. The well-characterized equations of motion, discrete time-stepping, and clear termination conditions for unrecoverable states enable the performance evaluator 126 to compute task-performance metrics such as episode duration and proficiency rates in a consistent and reproducible manner.

The closed-loop interface module 118 thereby enables the biological neural network 102 to exhibit goal-directed behavior through repeated interaction with the simulated task. At each control cycle, the encoder 120 transforms task state variables, such as pole angle and angular velocity, into electrical stimulation delivered to the input neural units 104, the decoder 122 converts electrical activity from the output neural units 106 into control signals that determine the applied force on the simulated cart, and the task updater 124 advances the task state accordingly. The performance evaluator 126 then derives a reward signal or task-performance metric based on how long the pole remains within the specified angular limits. Over many episodes, this closed-loop interaction allows the training module 128 to associate particular training electrical stimulation patterns delivered to the training neural units 108 with corresponding improvements or deteriorations in task performance.

In certain embodiments, the training module 128 implements an iterative selection process for the training electrical stimulation patterns that explicitly depends on the recent history of task performance. The training module 128 maintains value estimates for candidate training stimulation patterns, updates these value estimates based on changes in episode duration or other task-performance metrics observed after delivery of the patterns, and selects subsequent training stimulation patterns according to the updated value estimates. Because the value updates are driven by performance changes over recent episodes, the effectiveness of a given training pattern is state-dependent: a pattern that improves performance when the network is in one dynamical regime may have little effect or even reduce performance when the network occupies a different regime. By continuously re-estimating pattern values in real time, the training module 128 biases the selection toward training electrical stimulation patterns that, in the current network state, tend to increase episode duration and thus promote more stable control of the simulated task.

In some implementations, the biological neural network 102 achieves such goal-directed adaptation without relying on canonical in vivo reward pathways, such as dopaminergic neuromodulatory circuits. Instead, the reward signal is computed externally by the performance evaluator 126 from the behavior of the simulated task, and learning is induced by electronically delivered training electrical stimulation patterns applied to the training neural units 108. The biological neural network 102 thereby modifies its internal activity and connectivity in response to brief, structured electrical pulse trains that are timed and selected according to task-performance outcomes, rather than to endogenous neuromodulator release. This demonstrates that the closed-loop architecture of system 100 can systematically shape the information-processing capabilities of the biological neural network 102 to perform goal-directed control in an unstable dynamical environment using purely electronic interfaces for encoding, decoding, and training.

FIG. 1D illustrates an example stimulation schedule showing sequential activation of multiple stimulation electrodes during an experimental protocol. A first stimulation electrode 162 is driven with a train of biphasic electrical pulses over an initial time interval extending from a start time to approximately 25 seconds, as indicated along a time axis. A second stimulation electrode 164 is thereafter driven with a corresponding train of biphasic electrical pulses over a subsequent time interval extending from approximately 25 seconds to approximately 50 seconds. A third stimulation electrode 166 is then driven with a train of biphasic electrical pulses over a later time interval extending from approximately 50 seconds to approximately 75 seconds. The stimulation epochs for stimulation electrodes 162, 164, and 166 are non-overlapping in time and are shown as regularly spaced pulses to indicate periodic stimulation at a prescribed repetition rate. The schedule exemplifies how the system delivers stimulation to individual neural units in a temporally structured, electrode-specific manner during characterization and training, enabling isolation of stimulus-evoked responses associated with each stimulation site.

FIG. 1E illustrates a multi-organoid, multi-experiment workflow and training sequence implemented by the system. A first organoid 168, a second organoid 170, and a third organoid 172, along with additional organoids, are each assigned to a corresponding experiment 174, experiment 176, and experiment 178, respectively. Each experiment follows a standardized sequence of phases comprising a record phase 180, a stimulation phase 182, and a train or Cartpole phase 184. During the record phase 180, spontaneous activity of the organoid is acquired to identify putative neural units and establish baseline functional and causal connectivity. During the stimulation phase 182, the system delivers structured biphasic pulses to selected stimulation electrodes, for example as in FIG. 1D, to characterize stimulus-evoked responses and compute connectivity metrics used to configure input, output, and training neural units. During the train or Cartpole phase 184, the configured neural units are coupled in closed loop with a simulated dynamical environment implementing a cartpole task, such that the organoid receives task-encoding stimulation and returns control signals that influence the virtual environment.

The figure further depicts training organized into cycles, each cycle corresponding to a defined duration of closed-loop operation followed by a rest interval. A first cycle 186 represents a null condition in which no training stimulation is delivered, and the organoid interacts with the simulated environment without additional high-frequency training pulses. A second cycle 188 represents a random training condition in which training stimulation patterns are selected randomly from a set of candidate pulse sequences. A third cycle 190 represents an adaptive training condition in which training stimulation patterns are selected based on value estimates that are updated as a function of task performance. A fourth cycle 192 again represents a null condition, and additional cycles may be executed in various orders. Each cycle includes an active training period and a rest period, in which the organoid is not engaged with the cartpole environment, allowing recovery and assessment of longer-term state changes.

At a finer temporal scale, the figure illustrates individual episodes of the cartpole task, including an episode 1 designated by reference numeral 194 and an episode 2 designated by reference numeral 196. Within each episode, the simulated cartpole environment provides state information that is encoded into stimulation patterns delivered to input channels labeled input (left) and input (right), corresponding to input neural units receiving task-related electrical stimulation. The spike activity recorded from output neural units is decoded into control signals that determine the cart's horizontal actions until the episode terminates upon a failure event, such as the pole exceeding a terminal angle. Training channels labeled train i and train j represent training neural units that receive training electrical stimulation patterns. The schematic indicates that training pulses for train i and train j are delivered discretely at episode boundaries, for example following a failure, in accordance with the selected training paradigm for the corresponding cycle 186, 188, 190, or 192. In null cycles, no such training pulses are delivered; in random cycles, training patterns are sampled uniformly from a pool of sequences; and in adaptive cycles, training patterns are selected according to updated value estimates derived from recent changes in episode performance. The episodic representation thus demonstrates how the system coordinates encoding, decoding, environmental updates, and conditional delivery of training stimulation across multiple organoids, experiments, cycles, and episodes to induce and evaluate adaptive learning in the biological neural networks.

Organoid Generation and Interaction with In Vitro Learning

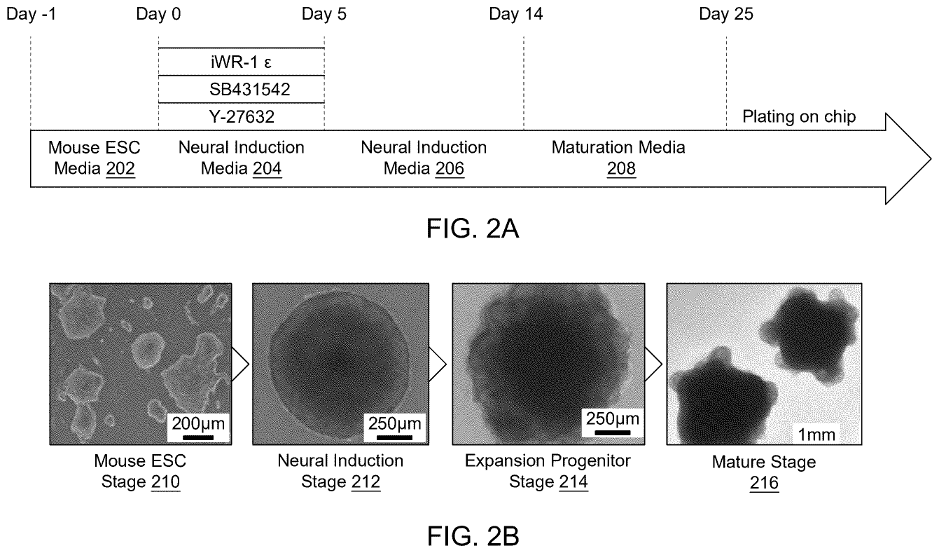

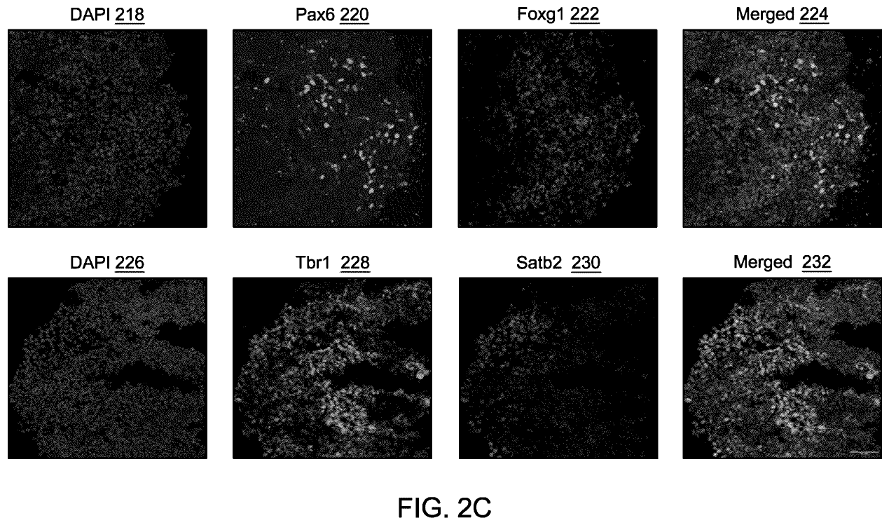

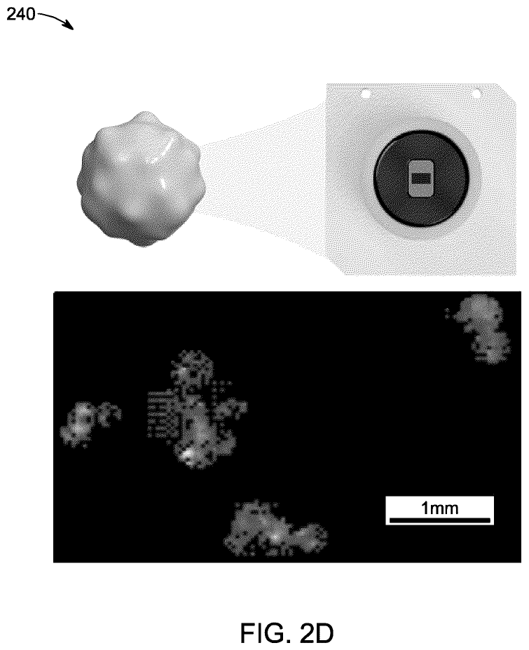

FIG. 2A illustrates a timeline for generation and maturation of mouse cortical organoids used as the biological neural network in the system. A horizontal axis indicates culture days from day −1 through day 25, with media transitions and small-molecule patterning cues annotated along the timeline. At day −1, embryonic stem cells grow in mouse ESC media 202. At day 0, the culture transitions to neural induction media 204 supplemented with small molecules including iWR-18, SB431542, and Y-27632, which direct the cells toward a cortical fate through directed patterning and self-organization. The neural induction media 206 continues through approximately day 5, maintaining conditions that support formation of three-dimensional aggregates. At approximately day 14, the culture transitions to maturation media 208, which supports neuronal differentiation, network formation, and synaptic maturation. Around day 25, the timeline indicates plating on chip, where the organoids are transferred onto a recording substrate for electrophysiological experiments. In some embodiments, the biological neural network comprises a cortical organoid derived from pluripotent stem cells, and the cortical organoid is cultured for a maturation period of about 20 days to about 50 days, such as about 30 days, to develop functional neural networks suitable for closed-loop experimentation.

FIG. 2B illustrates morphological stages of organoid development that correspond to the media and timing paradigm of FIG. 2A. A first panel labeled mouse ESC stage 210 depicts mouse embryonic stem cell colonies in mouse ESC media, with a scale bar of 200 μm. A second panel labeled neural induction stage 212 shows a spherical aggregate formed under neural induction media, with a scale bar of 250 μm, indicating the emergence of a three-dimensional neural induction structure. A third panel labeled expansion progenitor stage 214 shows an enlarged and textured spheroid with a scale bar of 250 μm, corresponding to an expansion of progenitor populations as the organoid grows. A fourth panel labeled mature stage 216 shows one or more larger organoids with a scale bar of 1 mm, representing a mature cortical organoid that has undergone directed patterning and self-organization to develop into structured neural tissue. Through directed patterning and self-organization, these three-dimensional aggregates develop from embryonic stem cells into structured neural tissue that recapitulates key features of cortical architecture, including radial organization and heterogeneous neuronal populations. In one embodiment, the biological neural network comprises a cortical organoid derived from pluripotent stem cells, and the cortical organoid develops functional neural networks within about 30 days of culture.

FIG. 2C illustrates immunohistochemistry and confocal imaging used to confirm cortical identity and laminar organization within the organoids. An upper row of panels shows staining at an early stage, around day 10, when the networks form forebrain-specified radial glial cells. A DAPI image 218 shows nuclear staining that delineates overall cell density. A Pax6 image 220 shows expression of the radial glial and progenitor marker Pax6, indicating a proliferative, forebrain-specified zone. A Foxg1 image 222 shows expression of the telencephalic marker Foxg1, confirming forebrain regional identity. A merged image 224 overlays DAPI, Pax6, and Foxg1 channels to highlight spatial co-localization of forebrain-specified radial glial cells. A lower row of panels shows staining at a later stage, around day 30, when the organoids mature to express subtype-specific markers. A DAPI image 226 again shows nuclear staining. A Tbr1 image 228 shows deep-layer excitatory neuron marker Tbr1. A Satb2 image 230 shows upper-layer excitatory neuron marker Satb2. A merged image 232 overlays DAPI, Tbr1, and Satb2 to demonstrate laminar-like organization with both deep and upper cortical layer neurons present. By day 10, the networks form forebrain-specified radial glial cells, and by day 30 they mature to express subtype-specific markers including upper (Satb2) and deep (Tbr1) layer neurons. In some embodiments, additional immunohistochemistry and confocal imaging further demonstrate the presence of inhibitory neurons expressing Sst and astrocytes expressing Gfap, with such results summarized in associated immunohistochemistry tables. These findings confirm that the organoids recapitulate key features of cortical architecture and justify the choice of cortical patterning due to the cortex's well-established role in adaptive information processing and its capability to encode, decode, and modify responses to novel inputs.

FIG. 2D illustrates interfacing of a cortical organoid with a high-density microelectrode array and the resulting spatial distribution of putative neuronal activity. An upper schematic 240 shows a mature cortical organoid positioned above and plated onto a microelectrode array chip. The enlarged view of the chip depicts a central electrode region onto which the organoid rests after plating on day 25 as indicated in FIG. 2A. In one embodiment, the organoids are interfaced with high-density microelectrode arrays (HD-MEA), providing precise spatio-temporal control over the culture with a high number of putative neuronal units available for potential computation. In particular, the biological neural network comprises a cortical organoid derived from pluripotent stem cells, and the multi-electrode array comprises a high-density microelectrode array configured to record from and to stimulate neural units at a surface of the cortical organoid. A lower panel in FIG. 2D shows a representative activity map with a scale bar of 1 mm, where grayscale patches indicate spatiotemporal footprints of putative neurons distributed across the electrode field. This panel illustrates that the HD-MEA captures activity from many spatially distributed units, enabling the system to characterize activity, identify putative neurons, compute spatio-temporal footprints, and later apply targeted stimulation and closed-loop training using the same high-density electrode platform.

In some embodiments, and as illustrated across FIGS. 2A and 2B, the directed patterning and self-organization of the mouse embryonic stem cells into cortical organoids result in three-dimensional aggregates that recapitulate key aspects of forebrain and cortical development. The sequential neural induction, progenitor expansion, and maturation stages establish radial glial-like scaffolds and proliferative zones from which differentiated neurons emerge and organize into layered configurations. During this process, the organoids develop spontaneous electrical activity and network-level dynamics consistent with early cortical circuit formation, providing a biologically realistic substrate on which adaptive information processing can be evaluated.

As further evidenced by the immunohistochemical characterization associated with FIG. 2C, the cortical organoids express a range of region- and layer-specific markers that indicate forebrain specification and cortical-like lamination. For example, Pax6- and Foxg1-positive populations are indicative of dorsal forebrain and cortical identity, while the presence of subtype-specific markers such as Satb2 and Tbr1 corresponds to upper- and deep-layer excitatory neurons, respectively. In some embodiments, additional staining (for example, for Sst-positive inhibitory interneurons and Gfap-positive astrocytes) confirms the emergence of inhibitory neuronal subtypes and glial support cells within the tissue. This molecular heterogeneity, together with the observed cytoarchitectural organization, supports the use of these organoids as a structurally enriched neural substrate for closed-loop interfacing.

In conjunction with FIG. 2D, the interfacing of the matured cortical organoids with high-density microelectrode arrays is configured to leverage this biological complexity for computation. The three-dimensional organoid tissue settles onto the planar electrode surface such that a subset of neurons and their processes lie in close apposition to the recording and stimulation sites. The high spatial density of electrodes enables simultaneous access to a large number of putative neural units distributed across distinct microdomains of the organoid, allowing the system to probe spontaneous and stimulus-evoked activity patterns that reflect contributions from multiple neuronal subtypes and layers. This configuration provides a rich set of candidate input, output, and training neural units for the adaptive learning paradigms described herein.

Accordingly, the combination of cortex-like laminar organization, diverse neuronal and glial cell types, and robust spontaneous and evoked activity renders cortical organoids a particularly suitable biological neural network for the disclosed system. The intrinsic heterogeneity and plasticity of these organoids enable the electrical interface and processing modules to encode task-related information, decode control-relevant signals, and apply training stimulation in a manner that exploits biologically grounded circuit dynamics, thereby enhancing the computational potential of the in vitro neural substrate.

### Neural Characterization and Connectivity Mapping

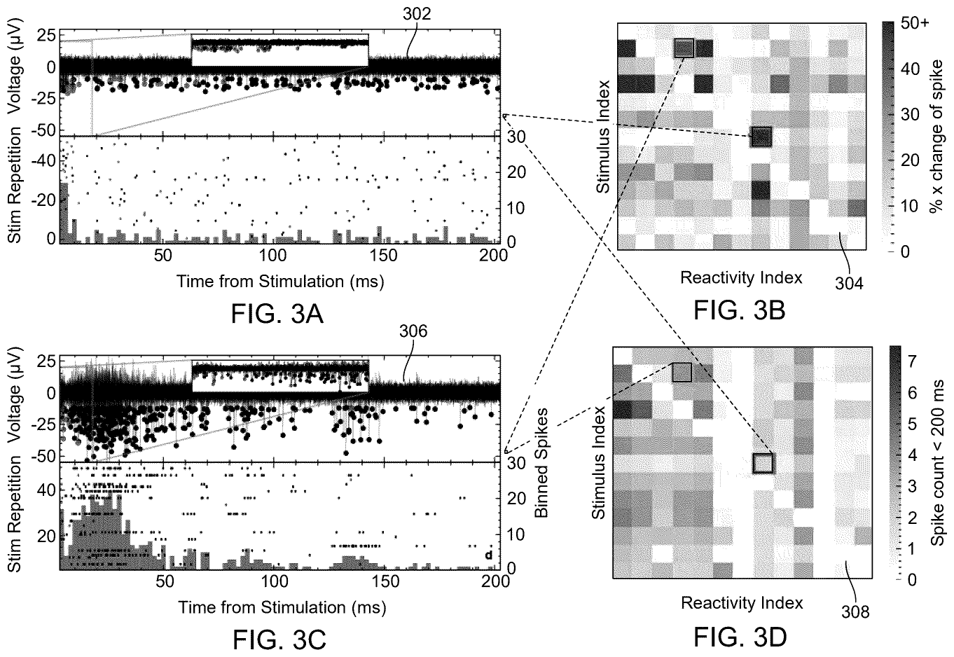

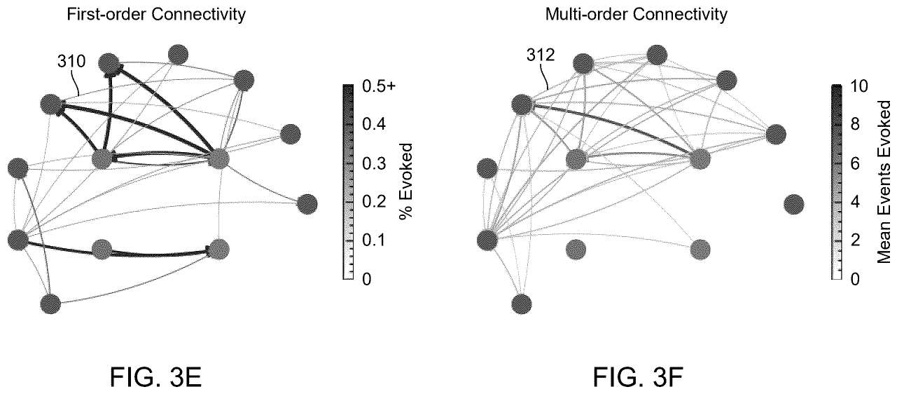

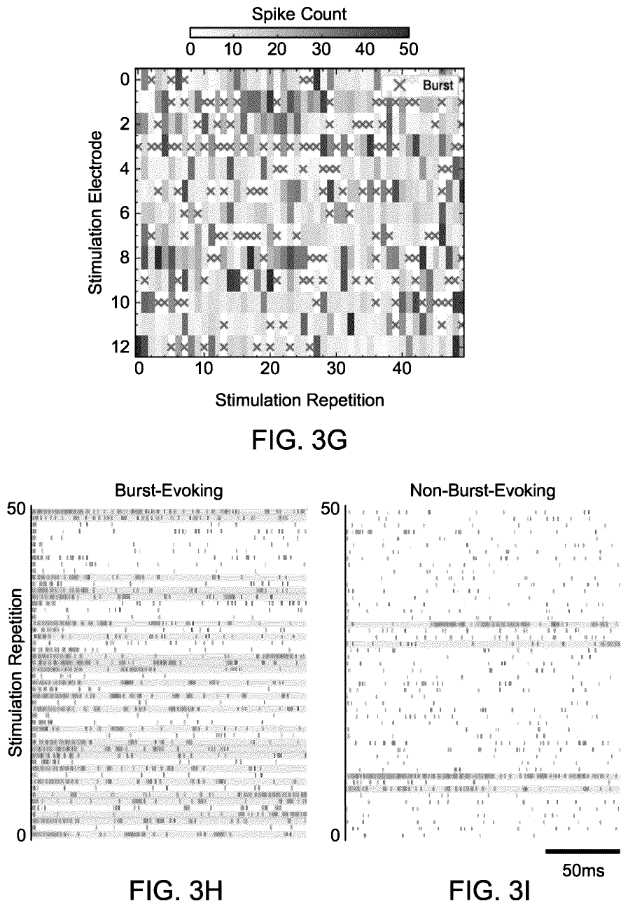

FIG. 3A illustrates stimulus-evoked responses obtained during characterization of stimulus-response relationships in the biological neural network. The top panel 302 shows overlapping voltage responses from multiple stimulation repetitions, each trace aligned to time from stimulation. These overlapping voltage responses represent the extracellular voltage fluctuations recorded from electrodes corresponding to identified putative neural units after bi-phasic electrical pulses are delivered to those units. The bottom panel of FIG. 3A shows a latency-from-stimulation raster combined with a latency histogram, in which each dot represents a detected spike and the histogram bins correspond to spike occurrences aligned to stimulus onset. This example highlights short-term responses, with the inset depicting the first 20 milliseconds following stimulation to emphasize short-latency activity that contributes to first-order causal connectivity metrics.

FIG. 3B illustrates a first-order causal connectivity heatmap 304 displaying the probability that a stimulus input evokes a reaction event within 18 milliseconds for the corresponding electrodes of interest. This heatmap quantifies the direct, first-order temporal response signature that represents the probability of direct stimulus-evoked action potentials within a first post-stimulus time window. Cells with higher intensity correspond to putative neural units exhibiting a higher proportion of evoked spikes at short latency. These first-order causal connectivity values provide directional connectivity information used to determine which putative neural units may serve as input neural units configured to receive electrical stimulation encoding task information and which may serve as output neural units configured to provide electrical activity for decoding control signals.

FIG. 3C illustrates stimulus-evoked responses similar to those of FIG. 3A but highlighting a multi-order bursting response. The top panel 306 shows overlapping voltage responses that include extended, network-wide activation occurring beyond the short-latency window. The bottom panel shows a latency raster and histogram that reflect sustained bursting activity following stimulation. This pattern corresponds to multi-order temporal signatures representing network-mediated responses in a longer post-stimulus time window and contributes to multi-order causal connectivity analysis. Units exhibiting frequent network-wide bursts are deemed less suitable as encoding units since widespread activation could interfere with more fine-grained control during task operation.

FIG. 3D illustrates a heatmap of multi-order causal connectivity 308 similar to FIG. 3B but showing mean response count within 200 milliseconds following stimulation. This heatmap quantifies the second temporal signature corresponding to multi-order causal connectivity representing network-mediated responses within a second post-stimulus time window that is longer than the first post-stimulus time window. Higher intensity in the heatmap indicates increased mean events evoked over the 200-millisecond window, providing information about the propensity of each putative neural unit to evoke downstream excitation across the network.

FIG. 3E illustrates first-order causal connectivity 310 represented as a directed network graph in which nodes correspond to putative neural units and edges indicate relative probabilities of direct evoked responses. The edge thickness reflects the strength of the first-order causal connectivity. FIG. 3E also illustrates chosen roles for an experiment, including selection of two encoding units and two decoding units, prioritizing pairs with strong first-order causal connectivity to maximize information transmission potential. These selections provide literal support for selecting a neural configuration comprising at least one input neural unit configured to receive electrical stimulation encoding task information and at least one output neural unit configured to provide electrical activity for decoding control signals. Units exhibiting strong first-order connectivity values exceeding predetermined thresholds are preferentially chosen to support closed-loop control.

FIG. 3F illustrates multi-order causal connectivity 312 similar to the representation of FIG. 3E but derived from multi-order temporal signatures showing network-mediated responses. Edges represent mean events evoked across the network rather than short-latency probabilities. Multi-order connectivity is used as a secondary selection criterion when determining the neural configuration for closed-loop experiments. Between 5 and 12 training units are selected independent of connectivity patterns to explore optimal training stimulations, and their selection is informed by the analysis shown in FIGS. 3A-3F.

The data represented in FIGS. 3A-3F are generated through a targeted approach to cortical organoid computation by focusing on characterizing capabilities within small sub-circuits. The approach begins with spontaneous activity in the record phase, which is used to identify locations of putative neurons and their corresponding electrodes to stimulate. Electrically stimulating the axon initial segment yields the best chance at triggering action potentials, and the spontaneous activity recording is used to generate a spatial map of putative neural unit locations through a metric incorporating firing rate and action potential amplitude. Signal averaging triggered by local maxima at these locations extracts spatio-temporal footprints for each unit, with larger amplitudes yielding neurons easier to identify in real-time experiments. Delivering bi-phasic pulses to each identified neural unit (50 pulses at 2 Hz) produces the stimulus-evoked responses shown in FIGS. 3A and 3C, enabling automated quantification of the three major temporal response signatures: first-order causal connectivity representing direct neural pathways, multi-order causal connectivity showing network-mediated responses, and probability of evoking network-wide bursts.

In some embodiments, and as illustrated in FIGS. 3A-3F, stimulus-evoked activity recorded during the stimulation phase enables the system to compute first-order causal connectivity values and multi-order causal connectivity values that more directly reflect directed information flow than conventional functional connectivity measures. The first-order causal connectivity values quantify probabilities of direct stimulus-evoked action potentials within a short post-stimulus time window, and the multi-order causal connectivity values quantify network-mediated responses within a longer post-stimulus time window. The peri-stimulus time histograms and reactivity heatmaps depicted in FIGS. 3A-3D provide a compact representation of these metrics across stimulation electrodes and recording electrodes, while the connectivity graphs in FIGS. 3E and 3F visualize how these directed pathways organize into sub-circuits suitable for encoding and decoding task information.

Analysis of experimental results demonstrates that these first-order causal connectivity values provide a substantially stronger prediction of downstream learning performance than functional connectivity metrics derived from spontaneous correlations alone. In trials that achieved high task proficiency, first-order causal connectivity between candidate input and output units exhibited markedly higher coefficients of determination with performance than did corresponding functional connectivity measures, indicating that the strength of direct, stimulus-locked pathways is a key determinant of effective control. Multi-order causal connectivity further complements this characterization by revealing how particular output units recruit broader network responses, which correlates with the ability of the biological neural network to generate stable, state-dependent control signals during closed-loop operation.

The configuration logic for selecting input neural units, output neural units, and training neural units leverages these causal connectivity values together with burst statistics derived from the same stimulus-response dataset. Input neural units are preferentially selected from putative neural units that exhibit robust, reliable first-order causal connectivity to downstream targets while maintaining a low probability of evoking network-wide bursts. In particular, the system determines, for each candidate stimulation site, a probability of evoking network-wide bursts defined as simultaneous action potentials detected at a majority of recording electrodes within a defined post-stimulus window, and it avoids using units with high burst probability as input neural units so that task-encoding stimulation does not trigger indiscriminate global activation. Output neural units are selected as putative neural units having first-order causal connectivity values from the selected input neural units that exceed a threshold probability value and that also display strong multi-order causal connectivity, reflecting their capacity both to respond sensitively to encoded task information and to recruit distributed network activity that supports motor-like output.

Training neural units are chosen from among remaining putative neural units that are distinct from the selected input neural units and output neural units, and, in some embodiments, they comprise between about 5 and about 15 training neural units. Because the adaptive training algorithms evaluate candidate training electrical stimulation patterns based on episode-wise changes in task performance, it is advantageous to select training neural units that span diverse microdomains within the organoid, even when their first-order causal connectivity values are weaker than those of the designated input and output units. By jointly considering first-order causal connectivity values, multi-order causal connectivity values, and the probability of evoking network-wide bursts, the characterization module and configuration module together define a neural configuration that maximizes information transmission potential while minimizing destabilizing burst responses, thereby enabling the closed-loop interface and adaptive training procedures to induce goal-directed learning in the biological neural network.

### Burst Characterization

FIG. 3G illustrates a burst characterization visualization 352 generated during the stimulation phase of the characterization procedure. For each stimulation electrode corresponding to a putative neural unit, the system delivers a plurality of biphasic stimulation pulses and records stimulus-evoked activity across the biological neural network over a post-stimulation analysis window. The burst characterization visualization 352 is rendered as a heatmap in which a horizontal axis represents stimulation repetition index, a vertical axis represents stimulation electrode index, and a color scale represents the total spike count within a defined analysis window, such as about 200 milliseconds following each delivered stimulation pulse. The characterization module computes, for each stimulation event, the total spike count across all monitored channels and classifies an event as a network-wide burst when the total spike count exceeds a threshold defined as a median spike count plus a multiple of a median absolute deviation, such as three median absolute deviations. Burst-classified events are overlaid on the heatmap as markers, for example red cross symbols, thereby indicating stimulation repetitions that evoke network-wide bursts. This visualization enables the system to quantify a probability of evoking network-wide bursts for each stimulation electrode and to compute burst-related metrics such as burstiness for use in selecting neural units and in determining multi-order causal connectivity, while allowing bursts to be excluded from certain connectivity calculations to focus on specific neural pathways.

FIG. 3H illustrates a burst-evoking raster representation 354 for stimulation conditions identified as burst-evoking in the burst characterization visualization 352. In the burst-evoking raster representation 354, each row corresponds to a stimulation repetition, a horizontal axis represents time relative to stimulation, and each point indicates an action potential detected on a recording channel. Time intervals classified as network-wide bursts are indicated by shaded regions extending across multiple channels and repetitions, thereby highlighting periods of dense, temporally clustered spike activity. The burst-evoking raster representation 354 demonstrates that certain stimulation electrodes and stimulation repetitions reliably produce large-scale, synchronized network responses characterized by high spike counts and rapid recruitment of multiple neural units. These data provide empirical support for the computed probability of evoking network-wide bursts and enable the system to identify stimulation sites that are unsuitable as input neural units because widespread activation could interfere with fine-grained encoding of task information.

FIG. 3I illustrates a non-burst-evoking raster representation 356 for stimulation conditions that do not meet the burst classification threshold. In the non-burst-evoking raster representation 356, each row again corresponds to a stimulation repetition and each point represents an action potential, but the spike activity is more sparsely distributed over time and across channels, and extended shaded burst regions are absent. The non-burst-evoking raster representation 356 visualizes stimulus-evoked responses that reflect localized or moderate network recruitment rather than network-wide bursts. These non-burst-evoking responses are used by the characterization module to compute first-order causal connectivity values representing probabilities of direct stimulus-evoked action potentials within a short post-stimulus time window and multi-order causal connectivity values representing network-mediated responses within a longer post-stimulus time window, while excluding burst periods from the multi-order connectivity calculation. In some embodiments, neural units that exhibit predominantly non-burst-evoking responses with strong first-order causal connectivity are preferentially selected as input neural units and output neural units, whereas neural units that frequently evoke bursts are avoided as encoding units and may instead be considered among the plurality of training neural units. Together with FIGS. 3A-3F, FIGS. 3G-3I thereby illustrate how stimulus-locked response statistics, burst detection, and causal connectivity analysis jointly inform selection of input neural units, output neural units, and training neural units for inducing adaptive learning in the biological neural network.

In some embodiments, selecting the at least one input neural unit comprises excluding putative neural units exhibiting burst-evoking probabilities that exceed a predetermined threshold. Network-wide bursts may be detected by computing, for each stimulation event, a total spike count across all recording channels and identifying events for which the count exceeds the median spike count plus three median absolute deviations. The burst probability of a putative neural unit is calculated as the proportion of stimulation repetitions for which the burst threshold is exceeded. Putative neural units demonstrating burst probabilities greater than approximately 0.5 are generally excluded from consideration as input neural units, as such units tend to evoke widespread network activation that may disrupt reliable encoding of task-state information. This exclusion criterion ensures that selected input neural units support stable information transmission without inducing global network perturbations that would confound decoding at the output neural units.

### Training Paradigms and Performance Under Cycled Training

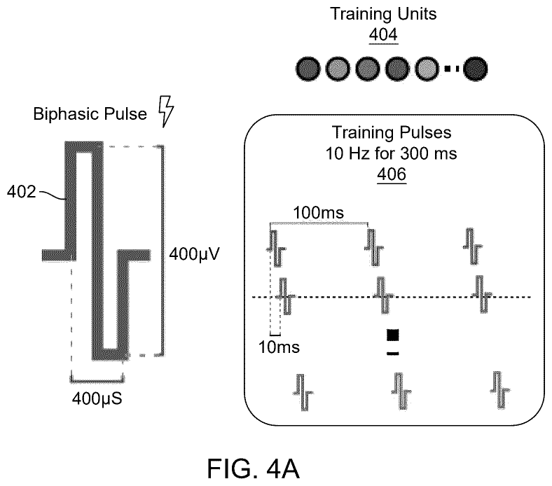

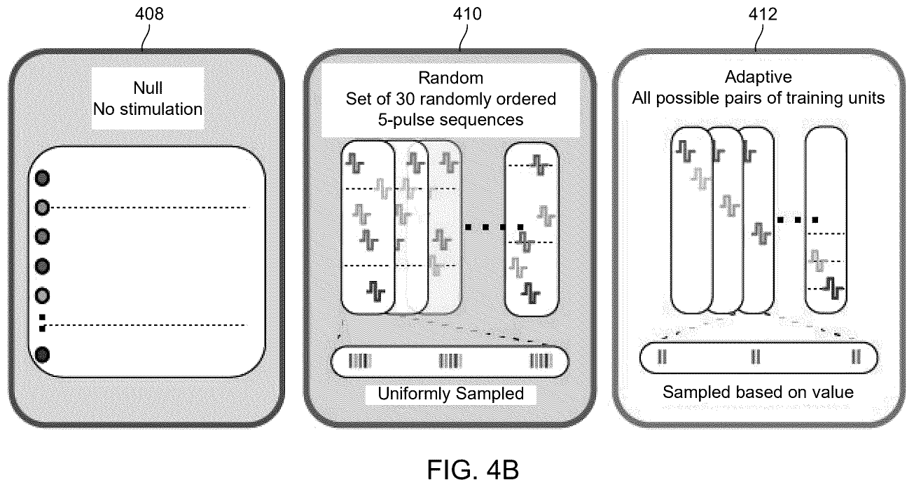

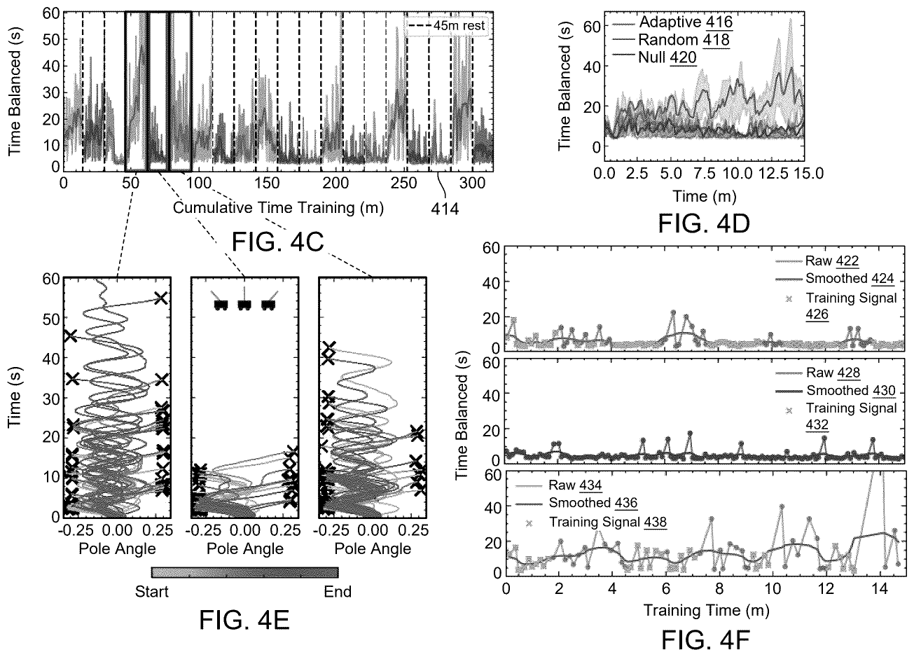

FIG. 4A illustrates training pulse parameters and delivery timing for training units. A left-hand panel 402 shows a square-wave biphasic pulse shape used for both characterization and training. The biphasic pulse has a 400 μV peak-to-peak amplitude and a 400 μs period, with the positive phase occurring first. A right-hand schematic shows a set of training units 404, each representing a training neural unit configured to receive training stimulation. A training pulses panel 406 illustrates that training pulses contain multiple pulses on separate channels spaced by 10 ms within each pulse pattern, repeating the pattern at 100 ms or 10 Hz. In the illustrated embodiment, the training pulses are delivered at 10 Hz for 300 ms, such that each training electrical stimulation pattern comprises a sequence of multiple biphasic electrical pulses delivered to one or more of the training units with inter-pulse intervals of about 10 ms and repeated at a repetition frequency of about 10 Hz for a duration of about 300 ms.

FIG. 4B illustrates three separate training paradigms implemented using the training units of FIG. 4A. A left panel labeled null 408 depicts a “Null” condition in which no stimulation is delivered to the training units, and episodes proceed without training pulses, thereby serving as a control. A central panel labeled random 410 depicts a “Random” stimulation condition using five-pulse patterns. In this condition, training pulses are organized into a set of 30 randomly ordered 5-pulse sequences, where the order of pulses in each sequence is randomly sampled from all possible training units and the sequences are uniformly sampled during training. A right panel labeled adaptive 412 depicts an “Adaptive” stimulation condition using value-optimized two-pulse patterns. In this condition, all possible pairs of training units define candidate two-pulse training patterns, and sampling is based on value rather than uniform probability. Adaptive selection of training electrical stimulation patterns is performed by maintaining value estimates for candidate training stimulation patterns, updating the value estimates based on changes in one or more task-performance metrics, and selecting subsequent training stimulation patterns according to the updated value estimates. In some embodiments, updating the value estimates comprises applying a temporal-difference learning algorithm with eligibility traces, including updating a value estimate V_{i,t} for a candidate training stimulation pattern i according to

$${V}_{i , t + 1} = {V}_{i , t} + α ( {R}_{t} - {V}_{i , t} ) {E}_{i , t} ,$$

where V_{i,t} represents a value estimate at time t, α represents a learning rate, Rₜ represents a reward signal based on task performance, and E_{i,t} represents an eligibility trace that is updated according to

$${E}_{i , t} = γ {E}_{i , t - 1} + {I}_{i , t} ,$$

where γ represents a decay factor and I_{i,t} indicates whether the candidate training stimulation pattern i was delivered.

FIG. 4C illustrates a cycled trial 414 showing performance, measured as time balanced in seconds, as a function of cumulative time training in minutes. The plot shows a cycled experiment where the training paradigm is cycled sequentially, for example Null→Random→Adaptive, with one condition per cycle. The training paradigm is indicated by color, such as blue for the null condition, red for the random condition, and green for the adaptive condition. Each cycle lasts 15 minutes, with a 45 minute rest between cycles, resulting in approximately 21 cumulative hours of experimentation. The figure shows that the adaptive condition repeatedly achieves superior performance, improving from a baseline of about 10 seconds to over 60 seconds of balanced control across multiple cycles, whereas the null and random conditions exhibit lower performance levels. The vertical dashed lines denote rest intervals between cycles. The performance traces in FIG. 4C thus illustrate a representative experiment in which targeted training signals modify information processing between input and output neurons and enable improvement of dynamic control behavior on the cartpole task.

FIG. 4D illustrates mean and inter-quartile range of performance per training paradigm for the same trial shown in FIG. 4C. The curves show time balanced in seconds as a function of time in minutes for the first 15 minutes of each condition. A curve 416 corresponds to the adaptive condition, a curve 418 corresponds to the random condition, and a curve 420 corresponds to the null condition. Shaded regions represent inter-quartile ranges, indicating variability across episodes. The cycle-averaged performance metrics in FIG. 4D quantify the improvement observed in FIG. 4C, with the adaptive condition exhibiting higher median and upper-quartile performance relative to the random and null conditions.

FIG. 4E is an enlarged view of selected portions of FIG. 4C, illustrating overlaid trajectories of pole angle throughout time within each training cycle for chosen cycles. The three panels in FIG. 4E show pole angle on the horizontal axis and time in seconds on the vertical axis, with trajectories from multiple episodes overlaid. Cross markers indicate episode termination when the pole exceeds a terminal angle, such as ±16° from vertical, which typically represents an unrecoverable state. A grayscale bar at the bottom of FIG. 4E indicates that color or shade can encode progression from the start to the end of each cycle. The emergence of effective control behavior becomes evident when examining the pole angle trajectories over time: under adaptive training, the trajectories remain closer to the upright position for longer durations before failure, reflecting improved stabilization compared with random or null conditions.

FIG. 4F illustrates plots of individual cycles, with each episode shown as scatter points and training delivery times shown for the relevant episodes. Three panels correspond to three example cycles, such as an adaptive cycle, a random cycle, and a null cycle. In the top panel, a raw performance trace 422 and a smoothed performance trace 424 show time balanced in seconds as a function of training time in minutes, while a training signal trace 426 marks episodes in which training pulses were delivered. In the middle panel, a raw performance trace 428, a smoothed performance trace 430, and a training signal trace 432 similarly depict another cycle. In the bottom panel, a raw performance trace 434, a smoothed performance trace 436, and a training signal trace 438 depict a third cycle. Training signals were delivered selectively at episode completion when short-term performance (5-trial mean) dropped below the longer-term average (20-trial mean). More generally, the system delivers a training stimulation pattern only when a short-term task performance metric calculated over a first number of recent task episodes falls below a long-term task performance metric calculated over a second number of recent task episodes that is greater than the first number of recent task episodes. FIG. 4F thus illustrates how training delivery times are aligned with performance decreases, implementing a conditional adaptive training rule that supports superior performance of adaptive training signals over random and null conditions.

In some embodiments, and as further illustrated in FIGS. 4A-4F, comparisons between the null, random, and adaptive training paradigms demonstrate that the adaptive selection of training electrical stimulation patterns yields superior task performance relative to both the absence of stimulation and randomly ordered stimulation patterns. When the training pulses are selected adaptively based on changes in the task performance metric, the time for which the simulated cartpole remains balanced progressively increases over successive cycles, as depicted by the rising envelopes of episode durations in FIGS. 4C and 4D. By contrast, in the null condition, episode durations tend to fluctuate around a relatively low baseline, and in the random condition, improvements are present but of smaller magnitude and reduced consistency. These observations indicate that high-frequency multi-neuron stimulation alone can modulate network dynamics, while performance-contingent adaptation of training patterns further enhances the ability of the biological neural network to acquire a stable control policy.

The temporal evolution of performance shown in FIGS. 4C, 4E, and 4F reflects that training effectiveness is state-dependent, meaning that the impact of a particular training electrical stimulation pattern depends on the recent activity history and current dynamical state of the biological neural network. As illustrated by episode-wise traces in FIG. 4F, the same training pattern can, in different cycles or different portions of a cycle, be associated with either improvements or decrements in episode duration. The value-estimation procedure implemented by the training module updates value estimates for candidate training stimulation patterns based on such observed changes in performance, and subsequent pattern selection probabilities are biased toward patterns that produce positive performance changes under the current network state. This adaptive reweighting enables the system to track and exploit transient windows in which specific training patterns are particularly effective, while de-emphasizing patterns whose effectiveness diminishes as the network reorganizes.

The pole-angle trajectories over time depicted in FIG. 4E provide further evidence that the biological neural network develops a coherent control policy under adaptive training. Early in training, the pole-angle traces exhibit scattered, rapidly diverging paths that frequently terminate at the episode boundary, indicative of unstable or poorly structured control. With continued adaptive stimulation, the trajectories increasingly cluster along paths that keep the pole near the upright position for extended periods before reaching terminal angles, indicating that the decoded control signals from the output neural units have become systematically tuned to the encoded state information. This progression from disorganized to structured pole-angle trajectories corresponds to the emergence of a task-specific mapping from encoded input stimulation frequencies to decoded control signals, evidencing that the biological neural network has undergone adaptive learning of the underlying dynamical control problem.

The multi-cycle structure of the experiments depicted in FIGS. 4C and 4D also reveals that adaptation occurs over multiple timescales. Within individual 15-minute training cycles, adaptive training pulses delivered at episode completion can induce relatively rapid shifts in performance, as indicated by abrupt increases in episode duration following certain training episodes. Across longer periods spanning multiple cycles separated by 45-minute rest intervals, the performance traces exhibit slower drifts, plateaus, and occasional regressions, suggesting that the biological neural network transitions between metastable network states that differentially support effective control. The training module, by conditioning training-pulse delivery on comparisons between short-term and long-term performance metrics, is configured to operate effectively across these timescales, providing rapid episode-level tuning while also accommodating slower, state-dependent reconfiguration of network dynamics.

Further analysis of firing-rate activity recorded at output neural unit channels across multiple experimental conditions revealed that adaptive training modulates output patterns in a performance-dependent manner rather than producing a generalized increase in network excitability. Baseline firing-rate distributions obtained during null, random, and adaptive conditions were comparable, exhibiting mean values between approximately 12 and 13 Hz with negligible pairwise effect sizes (Cohen's d<0.07). When evaluated at matched performance levels, however, distinct patterns emerged. At lower performance levels corresponding to episode durations less than approximately 10 seconds, output neural units under adaptive training showed reduced firing rates relative to null and random conditions. Conversely, at higher performance levels exceeding approximately 30 seconds, output neural units maintained elevated activity under adaptive conditions. Effect sizes increased monotonically with performance level, indicating that adaptive training reorganizes motor-related output activity to favor task-relevant firing configurations conducive to stable pole balancing.

The results summarized in FIGS. 4A-4F thus support the design of the adaptive training paradigm in which training electrical stimulation patterns are maintained as candidate patterns with associated value estimates, updates to the value estimates are computed based on changes in one or more task-performance metrics, and subsequent training electrical stimulation patterns are selected according to the updated value estimates. By demonstrating that such adaptive selection can systematically enhance performance on a continuously unstable dynamical control task, relative to null or randomly ordered training, these embodiments substantiate that the training module and its associated value-estimation and selection logic provide a technical mechanism by which a biological neural network cultured in vitro can be induced to exhibit goal-directed learning in a closed-loop control environment.

### Continuous Adaptive Training and Emergence of Control Policies

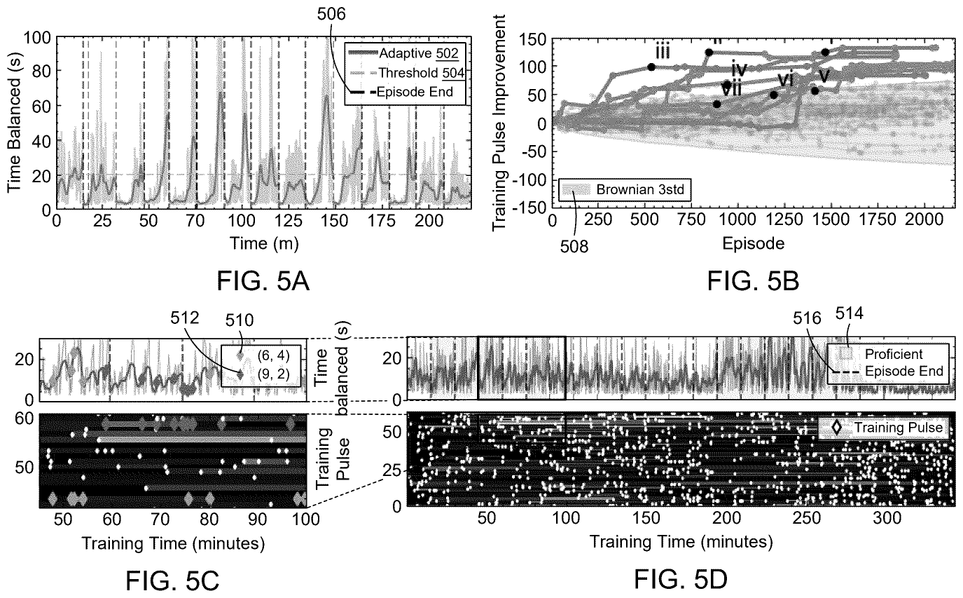

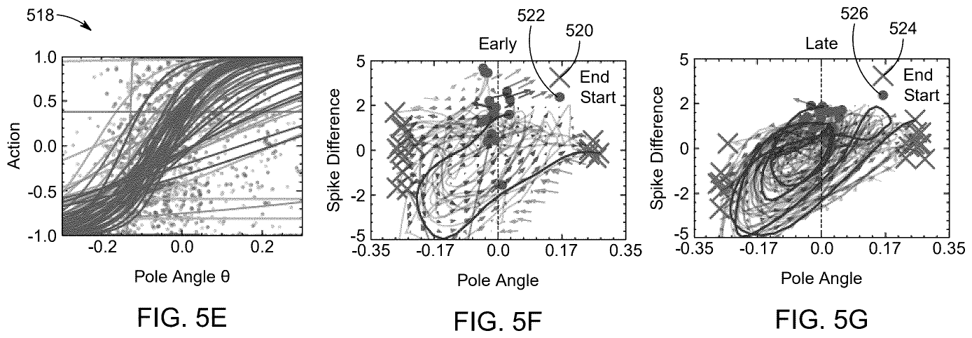

FIG. 5A illustrates performance of a biological neural network under an adaptive training paradigm operated continuously across multiple training cycles. The plot shows time balanced in seconds on the vertical axis versus training time in minutes on the horizontal axis. A trace 502 represents performance of the adaptive training paradigm running continuously for all cycles, without cycling between null and random conditions. A horizontal line 504 indicates a threshold of 20.5 seconds, which was designated as a “learner” threshold and corresponds to a proficiency threshold used to classify episodes or cycles as proficient when the task performance exceeds the predefined performance threshold. Vertical dashed lines 506 denote episode end times. In this continuous adaptive stimulation strategy, also referred to as continuous adaptive stimulation, performance remains consistently above the proficiency threshold for extended periods, demonstrating sustained learning across multiple hours with performance consistently exceeding the proficiency threshold. The temporal structure of performance shows clear autocorrelation, suggesting state-dependent changes in network behavior occurring over multi-hour periods. In broader experiments comparing training paradigms, adaptive stimulation significantly outperformed both random and null cases (p<XX, Holm-Bonferroni), whereas even random stimulation outperformed the null case, suggesting that high-frequency multi-neuron stimulations alone can modify network dynamics. While 22.8% of cycles reached proficiency under adaptive training, only 4.4% did so with random stimulation and 2.3% with no stimulation, and neural connectivity metrics strongly predicted performance outcomes, with both functional and causal connectivity showing significant correlations with proficiency.

FIG. 5B illustrates an improvement metric for various training pulses delivered under the continuous adaptive paradigm. The vertical axis represents training pulse improvement, measured as the cumulative change in time balanced following each training signal delivery, and the horizontal axis represents episode number. Each line corresponds to one candidate training pulse pattern, where line color denotes a first neuron in a training pulse and scatter color denotes a second neuron in the training pulse. The shaded envelope 508 represents Brownian motion bounds, specifically a three-standard-deviation envelope derived from a random walk model, which serves as a reference for improvements that could arise from stochastic fluctuations alone. Post-hoc analysis revealed that certain pulse combinations yielded consistently higher improvement metrics, measured as the cumulative change in time balanced following each training signal delivery, and these highly effective patterns—identified by improvement exceeding random walk bounds within or above the Brownian motion three-standard-deviation envelope—often shared common input neurons. These results indicate that adaptive training signals exploit specific connectivity motifs to drive performance improvements that cannot be explained by random drift.

FIG. 5C illustrates an inset and graph of performance through time with corresponding value estimation of training signals during continuous adaptive training. The lower portion shows training pulses arranged over training time, and the upper panel shows performance traces. The inset highlights two specific training pulses 510 and 512, illustrated for example in blue and purple, showing their estimated values changing through performance gain or loss after the pulses. With the blue pulse, a later pulse results in decreasing performance; thus, the value of said pulse is decreased correspondingly. Following principles from earlier work, a real-time value estimation method adaptively tracks the effectiveness of different training signals during each training session. In particular, adaptive selection of training electrical stimulation patterns is performed by maintaining value estimates for candidate training stimulation patterns, updating the value estimates based on changes in the one or more task-performance metrics, and selecting subsequent training stimulation patterns according to the updated value estimates. In some embodiments, updating the value estimates comprises applying a temporal-difference learning algorithm with eligibility traces, including updating a value estimate V_{i,t} for a candidate training stimulation pattern i according to

$${V}_{i , t + 1} = {V}_{i , t} + α ( {R}_{t} - {V}_{i , t} ) {E}_{i , t} ,$$

where V_{i,t} represents a value estimate at time t, α represents a learning rate, Rₜ represents a reward signal based on task performance, and E_{i,t} represents an eligibility trace that is updated according to

$${E}_{i , t} = γ {E}_{i , t - 1} + {I}_{i , t} ,$$

where γ represents a decay factor and I_{i,t} indicates whether the candidate training stimulation pattern i was delivered. The inset in FIG. 5C emphasizes how individual pulse patterns can drive either improvement or deterioration depending on the network's state, underscoring the importance of adaptive training signal selection.

FIG. 5D illustrates a longer-duration view of continuous adaptive training, combining performance traces with training pulse delivery times. The upper panel shows a green performance trace representing time balanced in seconds as a function of training time in minutes under continuous adaptive stimulation. A shaded band 514 indicates intervals in which performance exceeds the proficiency threshold and the organoid exhibits proficient control behavior. Vertical dashed lines 516 denote episode boundaries. The lower panel represents training pulses as points or diamonds aligned with training time and episode index, illustrating when training pulses are delivered relative to performance fluctuations. Training signals are delivered selectively at episode completion when a short-term task performance metric calculated over a first number of recent task episodes, such as a 5-trial mean, falls below a long-term task performance metric calculated over a second number of recent task episodes that is greater than the first number of recent task episodes, such as a 20-trial mean. The temporal pattern of performance peaks and training pulses suggests underlying state-dependent changes occurring over multi-hour periods, and the sustained prevalence of proficient intervals coincides with the development of a more refined control policy.

FIG. 5E illustrates sigmoid estimations 518 of the organoid's control policy through one training cycle. The horizontal axis represents pole angle θ, and the vertical axis represents action, for example a normalized control output derived from the decoded spike activity. Early episodes in the training cycle show scattered points and curves without cohesive structure, corresponding to an initial control policy that does not exhibit systematic dependence on pole angle. Late episodes approach a sigmoid centered around 0°, indicating that the organoid has developed a structured control policy that tends to push the cart in one direction when the pole angle is negative and in the opposite direction when the pole angle is positive. This simplified sigmoid policy estimation shows the emergence of structured control centered around the vertical position.

FIG. 5F illustrates early episodes 520 (for example, the first third of episodes) in terms of how the spike count difference between output units responds to input frequencies dependent on the cartpole's angle. The horizontal axis represents pole angle, and the vertical axis represents spike difference, for example the difference in smoothed firing rates between two output neural units. Vector flow lines 522 show the direction and magnitude of changes in spike difference as a function of state, mapping the pole's state to neural responses. These responses are short and show less coherent flow patterns, with end-state markers indicating failure points distributed asymmetrically across the state space. Early random responses thus lack a stable control strategy and do not yet encode a consistent mapping from pole angle to corrective action.

FIG. 5G illustrates late episodes 524 (for example, the last third of episodes) under continuous adaptive training, similar to the representation in FIG. 5F but after extended learning. The horizontal axis again represents pole angle, and the vertical axis represents spike difference. Flow fields and trajectories exhibit multiple oscillations and much higher density around 0°, and a cluster of trajectories 526 concentrates near an off-center balancing region that accounts for both angle and angular velocity. The complete input-output flow fields in FIGS. 5F and 5G map the pole's state to neural responses and show adaptation towards an off-center balancing point that accounts for both angle and angular velocity. Early episodes show scattered, inconsistent responses, but late episodes demonstrate coherent control strategies with multiple stable oscillation patterns and increased activity density near this preferred balancing state. This improvement coincides with the development of a more refined control policy under continuous adaptive training, where early random responses evolve into structured state-dependent control. Adaptive selection of the training electrical stimulation patterns therefore achieves a higher fraction of episodes exceeding a proficiency threshold than random selection of training electrical stimulation patterns or operation without training electrical stimulation patterns, consistent with the superior performance of adaptive training signals over random and null conditions.

In some embodiments, and as further illustrated in FIGS. 5A-5G, the training module is configured to implement a stochastic value-estimation process that tracks the effectiveness of candidate training electrical stimulation patterns over extended periods of continuous adaptive operation. Because episode durations are inherently variable due to ongoing fluctuations in the biological neural network, the value estimates associated with individual training patterns are updated using reward signals derived from changes in the time-balanced performance metric over successive episodes and are filtered through eligibility traces that weight recently delivered patterns more strongly than patterns delivered in the distant past. In this way, the value-estimation mechanism accounts for both short-term variability and longer-term performance trends, allowing the system to assign credit or blame to particular training stimulation patterns despite stochasticity in episode outcomes.

The continuous adaptive experiments depicted in FIGS. 5A and 5B further demonstrate that performance under adaptive training exhibits temporal autocorrelation across episodes and cycles, consistent with multi-timescale adaptation of the biological neural network. Episodes with high time-balanced values tend to cluster in contiguous temporal segments, indicating that once the biological neural network enters a favorable dynamical regime, it can sustain effective control behavior over multiple subsequent episodes before drifting into less favorable regimes. This temporal structure is reflected in the gradual upward trends in training-pulse improvement metrics across episodes in FIG. 5B and in the persistence of proficient performance segments in FIGS. 5C and 5D, and is distinct from the behavior expected from a memoryless or purely random process. These observations support that the training signals induce lasting, state-dependent modifications to the network that extend beyond the immediate episode in which a particular training pattern is delivered.

The input-output relationships illustrated in FIGS. 5E-5G show that adaptive stimulation guides the biological neural network from an early regime characterized by scattered, weakly structured responses toward a late regime in which the decoded control actions form a coherent, state-dependent policy. During early episodes, the mapping between pole angle and decoded action exhibits substantial dispersion, and the spike-difference flow fields are diffuse, indicating that small changes in input encoding do not reliably translate into consistent output control signals. With continued continuous adaptive training, the action-versus-angle curves progressively sharpen toward sigmoidal profiles centered near a preferred operating region, and the flow fields in FIGS. 5F and 5G develop organized trajectories with increased vector density around a specific balancing region in the joint space of pole angle and spike-rate difference. These patterns indicate that the biological neural network has learned a control policy that preferentially stabilizes the system near a particular, potentially off-center, balancing point that jointly accounts for instantaneous pole angle and its recent evolution.

In some embodiments, the continuous adaptive training paradigm is further validated through pharmacological perturbation of synaptic transmission within the biological neural network. In such experiments, cortical organoids that have previously achieved stable performance on the cartpole task under the adaptive training paradigm are exposed to a combination of glutamatergic receptor antagonists, such as an AMPA receptor antagonist (NBQX) together with an NMDA receptor antagonist (APV). These compounds are applied to the culture medium while the closed-loop control system continues to operate, so that changes in task performance can be monitored continuously during blockade of fast excitatory synaptic transmission and during subsequent washout.

Prior to drug application, the organoids exhibit repeated episodes of successful balancing, with multiple episodes achieving time-balanced durations within the upper decile of performance for the corresponding training cycle. During administration of NBQX and APV, the time-balanced performance metric collapses toward near-zero values, and episodes that reach the upper performance decile become rare or absent, indicating a loss of effective control of the inverted pendulum. Following removal of the antagonists and restoration of standard maturation media, the organoids progressively recover their ability to balance the pole, with the time-balanced durations returning toward, and in some cases exceeding, pre-drug values. In certain implementations, performance is quantified by normalizing the 90th percentile of episode durations at different times to the 90th percentile of a reference early cycle, demonstrating a marked reduction during pharmacological blockade and a gradual increase to or above baseline following washout.

These pharmacological experiments indicate that the learned control behavior depends on intact glutamatergic synaptic transmission and is not solely a consequence of the stimulation protocol or the task-side dynamics. The reversible suppression and recovery of performance support the conclusion that the adaptive training electrical stimulation patterns engage biological learning mechanisms within the cortical organoids, rather than producing fixed, non-plastic responses. The results further suggest that the training module, which adaptively selects training electrical stimulation patterns based on task performance, interacts with synaptic and network-level processes that operate over multiple timescales, thereby enabling the biological neural network to acquire, transiently lose, and re-establish effective control policies for the dynamical task.

In certain embodiments, the system further enables investigation of biological mechanisms underlying task-directed learning by administering pharmacological agents during closed-loop operation. In illustrative experiments, glutamatergic receptor antagonists targeting AMPA/kainate and NMDA receptor pathways were introduced to evaluate their contribution to adaptive control behavior. Application of NBQX at approximately 20 μM and APV at approximately 100 μM resulted in a marked impairment in task performance, with organoids exhibiting substantially reduced episode durations relative to pre-drug baselines. Across a plurality of organoids, mixed-effects statistical modeling indicated that drug administration reduced performance by approximately 10.38 seconds (95% CI: −13.52 to −7.25; p<0.001), corresponding to an approximate 64% decrease in balanced-pole duration irrespective of initial performance levels. Following washout of both antagonists, performance recovered toward baseline, with a residual deficit of approximately 1.51 seconds (95% CI: −4.48 to 1.45; p=0.318), demonstrating that the functional impairment was reversible. These results indicate that glutamatergic neurotransmission mediated through AMPA/kainate and NMDA receptors is necessary for sustaining the goal-directed behavioral adaptation supported by the disclosed closed-loop system.

Collectively, the continuous adaptive results associated with FIGS. 5A-5G demonstrate that the adaptive selection and stochastic value estimation of training electrical stimulation patterns not only improve aggregate performance but also drive the emergence of structured, reproducible control policies in the biological neural network over multi-hour training intervals. The presence of autocorrelated performance segments, the progressive refinement of action-angle mappings, and the convergence of spike-difference dynamics toward a stable balancing region all provide evidence that the disclosed closed-loop interface and training framework support genuine goal-directed learning in vitro, rather than transient or purely reactive modulation of neural activity.

### Comparative Outcomes and Connectivity Predictors of Learning

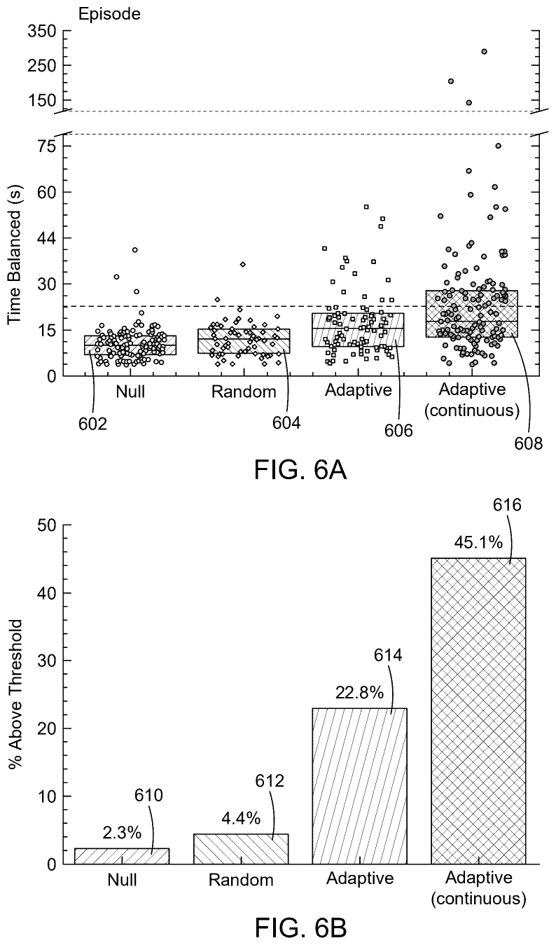

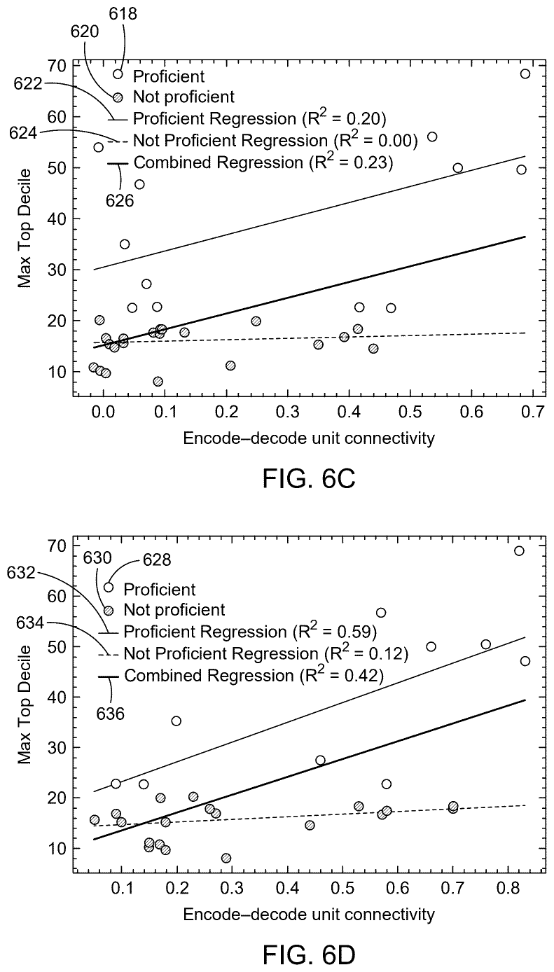

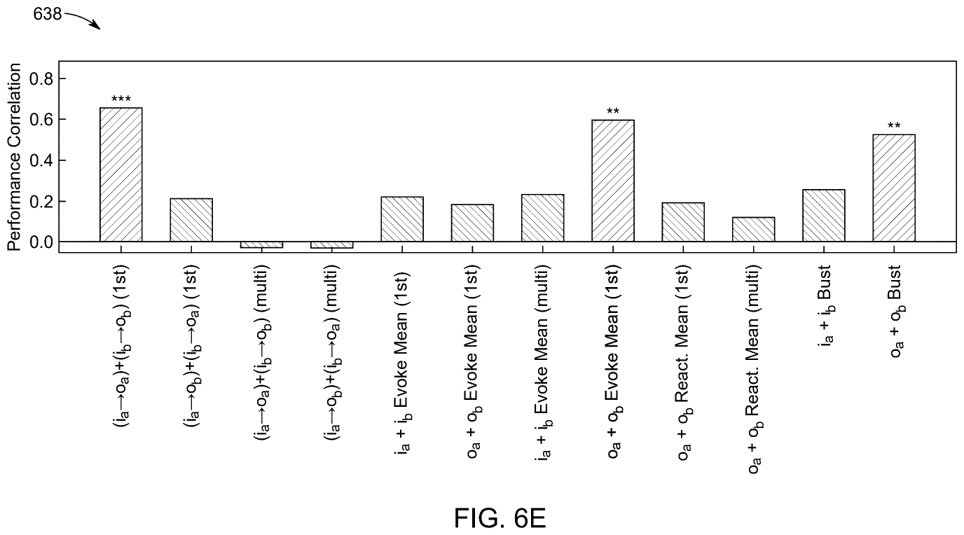

FIG. 6A illustrates performance distributions for different training paradigms. The horizontal axis lists experimental conditions, including a null condition 602 with no stimulation, a random stimulation condition 604 using five-pulse patterns, an adaptive condition 606 with value-optimized training stimulation, and an adaptive (continuous) condition 608 in which the system applies adaptive training continuously across cycles rather than cycling with other conditions. Each data point within a box plot represents the 90th percentile performance within a cycle, measured as time balanced in seconds on the vertical axis. The box plots show the inter-quartile range, and whiskers show broader variability across cycles. A horizontal dashed red line indicates a threshold of about 20.5 seconds, which defines a “proficient” level of task performance and corresponds to a proficiency threshold used to determine when task performance exceeds a predefined performance threshold. The adaptive condition 606 and the adaptive (continuous) condition 608 show a larger fraction of cycles with 90th percentile performance above the proficiency threshold than the null condition 602 and random 604 conditions. Several cycles under adaptive (continuous) training achieve time balanced values well above 75 seconds, and some episodes extend towards 350 seconds, consistent with sustained control behavior.

FIG. 6B illustrates the percentage of proficient cycles above the proficiency threshold for each training paradigm. The vertical axis shows the percentage above threshold, and the horizontal axis lists the four conditions. A first bar 610 corresponds to the null condition and indicates that about 2.3% of cycles reach proficiency. A second bar 612 corresponds to the random condition and indicates that about 4.4% of cycles reach proficiency. A third bar 614 corresponds to the adaptive condition and indicates that about 22.8% of cycled adaptive trials reach proficiency. A fourth bar 616 corresponds to the adaptive (continuous) condition and indicates that about 45.1% of trials achieve proficiency. From these training paradigms, the adaptive training paradigm significantly outperformed both random and null cases (p<0.001, Holm-Bonferroni corrected). This effect strengthened further in continuous adaptive experiments, where 45.1% of trials achieved proficiency and significantly outperformed all other conditions (p<0.001). These results demonstrate both the effectiveness of adaptive training and the importance of delivering training electrical stimulation patterns based on task performance rather than using random or null stimulation.

FIG. 6C illustrates how functional connectivity predicts high-end performance across trials. The horizontal axis represents encode-decode unit connectivity, calculated as a functional connectivity metric in the baseline spontaneous recording between input and output units used for encoding task information and decoding control signals. The vertical axis represents “Max Top Decile,” which corresponds to the 90th percentile performance for each trial. Red markers 618 denote proficient trials that exceed the proficiency threshold, and blue markers 620 denote not proficient trials that do not reach the threshold. A dashed red line 622 represents a regression fit for proficient trials, with coefficient of determination R²=0.20. A dashed blue line 624 represents a regression fit for not proficient trials, with R²=0.00. A dashed black line 626 represents a combined regression fit for all trials with R²=0.23. Functional connectivity calculated in the baseline recording thus correlates with 90th percentile performance (R²=0.23, p<0.01), but the predictive strength remains modest, particularly for not proficient trials.

FIG. 6D illustrates a similar analysis using a first-order causal connectivity metric instead of functional connectivity. The horizontal axis again represents encode-decode unit connectivity, but now derived from first-order causal connectivity values representing probabilities of direct stimulus-evoked action potentials occurring within a first post-stimulus time window following stimulation. The vertical axis again represents Max Top Decile performance. Red markers 628 again denote proficient trials, and blue markers 630 denote not proficient trials. A dashed red line 632 represents a regression fit for proficient trials with R²=0.59. A dashed blue line 634 represents a regression fit for not proficient trials with R²=0.12. A dashed black line 636 represents a combined regression fit with R²=0.42. The first-order causal connectivity metric proves especially predictive of performance outcomes, with R²=0.42, p<0.001, substantially outperforming the functional connectivity metric with R²=0.23, p<0.01. This advantage is most pronounced in proficient trials, where causal connectivity shows a remarkably strong correlation with performance (R²=0.59) compared to functional connectivity (R²=0.20). The strength of first-order causal connections, which guides neural configuration selection of input and output neural units, therefore emerges as a key predictor of learning capability.

FIG. 6E illustrates a correlation analysis 638 between multiple connectivity features and 90th percentile performance. The vertical axis represents performance correlation, for example a correlation coefficient between each feature and the 90th percentile performance. The horizontal axis lists different connectivity features constructed from combinations of input units i and output units o, including first-order connectivity terms such as (iₐ→oₐ)+(i_{b}→o_{b})(1st), cross-terms such as (iₐ→o_{b})+(i_{b}→oₐ)(1st), multi-order connectivity measures such as (iₐ→oₐ)+(i_{b}→o_{b})(multi) and (iₐ→o_{b})+(i_{b}→oₐ)(multi), evoked mean responses such as iₐ+i_{b} evoke mean (1st) and oₐ+o_{b} evoke mean (1st), multi-order evoked means such as iₐ+i_{b} evoke mean (multi) and oₐ+o_{b} evoke mean (multi), reaction means such as oₐ+o_{b} react. mean (1st) and oₐ+o_{b} react. mean (multi), and burst-related metrics such as iₐ+i_{b} burst and oₐ+o_{b} burst. Bars drawn in green highlight features with statistically significant correlations, whereas black bars denote weaker or non-significant correlations. The combined first-order input-output connectivity feature (iₐ→oₐ)+(i_{b}→o_{b})(1st) exhibits the strongest correlation, reaching a performance correlation above 0.6 and marked with (***) to indicate high significance. Output units' ability to evoke multi-order responses and network-wide bursts, represented by features such as oₐ+o_{b} evoke mean (multi) and oₐ+o_{b} burst, shows significant correlations with performance (marked with **, and associated p<0.01), suggesting that output units' capacity to recruit broader network activity may facilitate adaptive control. Other connectivity metrics, including burst probability and functional coupling between non-input/output units, show weaker or non-significant correlations with R²<0.1, which further supports the importance of first-order causal pathways between input and output units in enabling successful learning.

The disclosed closed-loop electrophysiology framework described through FIG. 1 through FIG. 6 enables task-specific benchmarking of biological neural networks embodied in a simulated dynamical environment. The framework provides logic to characterize a network by recording spontaneous activity to detect putative neural spatiotemporal footprints, logic to utilize targeted neural stimulation to identify peri-stimulus time histograms and calculate causal connectivity, logic to use the characterization of individual neural units to select a neural configuration involving input, output, and training neurons, and logic to evaluate the network on a simulated inverted pendulum problem, such as the cartpole task.

In some embodiments, mouse cortical organoids serve as a biological substrate for learning. These organoids self-organize into three-dimensional, layered tissue that recapitulates key features of cortical architecture and develop functional neural networks within about thirty days, as illustrated in FIGS. 2A and 2B. Directed patterning and maturation media drive the emergence of forebrain-specified radial glial cells and post-mitotic excitatory neuronal subtypes together with inhibitory neurons and astrocytes, as shown by immunohistochemistry in FIG. 2C. The organoids interface with high-density microelectrode arrays as depicted in FIG. 2D, which provide precise spatio-temporal control and readout from a high number of putative neuronal units suitable for computation.

The framework implements a multi-phase experimental approach as schematized in FIGS. 1B and 1C. A record phase acquires spontaneous neural activity and performs automated analysis to locate neurons based on the quantity and magnitude of action potentials above a predefined threshold. From these recordings, a spatial map of putative neural unit locations is generated and a metric that combines normalized log spike rate and mean spike amplitude identifies electrodes with reliable spiking. Spatio-temporal footprints are extracted for each unit, which facilitates robust detection of neural activity on distinct electrodes during subsequent phases.

A stimulation phase then characterizes stimulus-response properties of the identified units. Biphasic electrical pulses are delivered to each unit, and the network response is captured over multiple temporal windows, as illustrated in FIGS. 3A and 3C. From a response tensor constructed around each stimulus event, first-order connectivity values quantify the probability that stimulation at a given electrode evokes a spike within a short latency window, and multi-order connectivity values quantify the average number of spikes over a longer window. Network-wide bursts are detected by thresholding the total spike count across channels and excluded from certain calculations to emphasize specific pathways. Heatmaps of first-order and multi-order causal connectivity, exemplified in FIGS. 3B and 3D, reveal both direct and network-mediated connections. These connectivity measures support selection of encoding units, decoding units, and training units, as shown in FIGS. 3E and 3F, where strong first-order pathways are prioritized for information transmission while units prone to frequent bursting are avoided as encoders.

Using this characterized neural configuration, the framework conducts closed-loop training in a cartpole environment that embodies an inverted pendulum control problem, as diagrammed in FIG. 1C. The cartpole system state includes cart position, cart velocity, pole angle, and angular velocity. At each discrete timestep, a force in a bounded range, such as between −10 N and 10 N, is applied to the cart. Episodes begin from small perturbations of the pole angle and angular velocity and terminate when the pole angle exceeds a terminal value, such as about ±16 degrees, representing an unrecoverable state.

Information exchange between the organoid and the virtual environment uses rate-coding. Two input neurons receive stimulation frequencies determined by the instantaneous pole angle such that the frequencies diverge in opposite directions as the pole tilts away from vertical while remaining in a biologically relevant range. Two output neurons generate spikes that are converted into smoothed firing rates by exponential filtering. The difference between the smoothed firing rates of the output units determines the direction and magnitude of the force applied to the cart. Within each timestep of the real-time loop, the system reads motor neuron activity for a predefined read window, decodes the motor signal into a force, updates the cartpole state, encodes the new state into updated stimulation frequencies, and, when appropriate, enters a training phase in which training pulses are delivered and value estimates are updated. Precise millisecond-scale timing minimizes latency between the neural culture and the environment and yields stable yet responsive control.

The framework evaluates multiple training paradigms that differ in how training signals are selected and applied, as summarized in FIGS. 4A and 4B. In a null condition, no stimulation is given. In a random condition, training signals consist of five-pulse patterns in which sequential biphasic pulses traverse randomly sampled training electrodes. In an adaptive condition, training signals consist of value-optimized paired pulses selected according to a reinforcement-learning-style rule that updates a value estimate for each electrode based on observed changes in performance and maintains an eligibility trace that credits recently used pulses. Selection probabilities are proportional to these value estimates subject to a lower bound. Training signals are delivered conditionally when a short-term performance measure, such as a five-episode mean of time balanced, drops below a longer-term moving average, such as a twenty-episode mean. Representative training cycles in FIGS. 4C-4F show that adaptive training repeatedly elevates performance above baseline, yields longer periods of stable balancing, and aligns delivery of training pulses with episodes of declining performance.

When tested across multiple organoids and experiments, adaptive training pulses significantly outperform random and null conditions. Box plots in FIG. 6A compare the ninety-th percentile time-balanced performance per cycle under null, random, adaptive, and continuous adaptive conditions, with a proficiency threshold indicated. FIG. 6B shows the percentage of cycles exceeding this threshold, where adaptive trials and continuous adaptive trials achieve higher proficiency rates than random or null trials. Across experiments, adaptive training achieves proficiency in a substantial fraction of cycled trials and nearly half of continuous adaptive trials, demonstrating that biological neural networks can be systematically modified through precise electronic control. Even random stimulation improves performance relative to no stimulation, indicating that high-frequency multi-neuron stimulations alone can reshape network dynamics.

Connectivity analysis further reveals how the selected neural configuration influences learning capability. Scatter plots in FIGS. 6C and 6D demonstrate that functional connectivity and first-order causal connectivity calculated from baseline recordings correlate with ninety-th percentile performance, with first-order causal connectivity showing stronger predictive power, particularly in proficient trials. FIG. 6E presents the correlation of multiple connectivity-derived features with performance; metrics that capture the strength of first-order causal pathways between input and output units, and the ability of output units to evoke multi-order responses and network-wide bursts, exhibit the highest correlations. These findings indicate that effective neural interfaces benefit from neurons capable of recruiting broader network activity via direct stimulus-evoked pathways, and that first-order causal connectivity provides a key predictor for selecting neural configurations that support successful learning.

To further examine learning under sustained exposure to adaptive training signals, the framework executes continuous adaptive experiments in which only the adaptive paradigm operates across many cycles, as shown in FIGS. 5A-5G. Time-series plots of performance in FIG. 5A demonstrate sustained learning over multiple hours with performance consistently above the proficiency threshold. Improvement metrics for individual training pulses in FIG. 5B show that certain pulse combinations repeatedly yield large positive changes in time balanced, and these combinations often share common input neurons. FIG. 5C illustrates real-time value estimation of training signals, where the estimated value of a specific pulse pattern increases when it precedes performance gains and decreases when later deliveries of the same pattern correlate with poorer performance, highlighting the state-dependent nature of training effectiveness. Policy estimation and flow-field visualizations in FIGS. 5E-5G depict how early episodes exhibit scattered, incoherent control, whereas late episodes converge toward a structured control policy centered near a preferred off-center balancing point with multiple stable oscillatory trajectories around the vertical state.

The overall framework is implemented in a python-based platform referred to as the BrainDance platform. The platform supports flexible specification of experimental phases, real-time signal processing, and online adjustment of training policies, thereby enabling rapid iteration akin to the prototyping cycles used in artificial neural network development. The platform integrates high-density planar MEA recordings, rate-based encoding and decoding, and adaptive training algorithms. Present implementations primarily access neurons located at the organoid surface in contact with the array and use single-electrode thresholding to extract spikes, which may include multi-unit contributions. Future implementations can extend the framework by incorporating local field potential readouts, volumetric recording modalities, and automated neural role assignment based on latent-space representations of neural activity.

The materials and methods underlying this framework establish reproducible preparation of the biological and electronic components. Mouse embryonic stem cells are maintained under defined culture conditions that include vitronectin-coated substrates, serum-supplemented maintenance medium, appropriate amino acid, pyruvate, glutamine, antioxidant, and antibiotic components, and leukemia inhibitory factor to sustain pluripotency. Cortical organoids are generated by single-cell dissociation of embryonic stem cells, re-aggregation in low-adhesion wells with Rho kinase inhibition, staged transitions through cortical differentiation medium and neuronal differentiation medium on orbital shakers, and subsequent maturation in neuronal maturation medium containing defined supplements and reduced-growth-factor matrix. These temporal media changes and growth conditions correspond to the schematic stages and bright-field images shown in FIGS. 2A and 2B. Immunohistochemistry and confocal imaging protocols define fixation, cryoprotection, sectioning, blocking, primary and secondary antibody panels, nuclear counterstaining, and imaging settings that yield the fluorescence images in FIG. 2C.

Organoids are plated on high-density MEA chips after sequential coatings with poly-L-ornithine, laminin, and fibronectin to promote attachment, as depicted in FIG. 2D. Electrophysiology uses the MaxOne recording system to acquire extracellular signals at high sampling rates across hundreds to thousands of channels, while limiting the number of simultaneously active stimulation electrodes and constraining recording duration to maintain thermal stability. Real-time signal processing removes artifacts and applies spike-detection thresholds expressed in multiples of the noise standard deviation or root-mean-square value.

During experiments, the closed-loop system advances in discrete timesteps. A read phase monitors output neuron activity and updates smoothed firing rates. An environment-update phase decodes the motor command, advances the physics of the cartpole environment, and encodes the new state into updated input stimulation rates. A training phase operates conditionally at episode completion, delivers training pulses according to the selected paradigm, and updates value estimates for the electrodes. The timing of these phases balances the need for accurate spike-rate estimation with the desire for responsive control, thereby preserving real-time interaction between the cortical organoid and the simulated dynamical environment.

In some embodiments, and as further illustrated by FIGS. 6A-6E, comparative analysis across the null, random, cycled adaptive, and continuous adaptive training conditions demonstrates that adaptive training yields statistically significant improvements in task performance and proficiency rates. Boxplot representations of cycle-wise performance indicate that the distributions corresponding to adaptive conditions, particularly continuous adaptive operation, are shifted upward relative to the null and random conditions, with a larger proportion of cycles exceeding a defined proficiency threshold based on high-percentile episode durations. Statistical testing using multiple-comparison corrections (for example, Holm-Bonferroni procedures) confirms that adaptive training conditions differ significantly from both random stimulation and no-stimulation baselines, thereby substantiating that the observed performance gains are not attributable to chance fluctuations in network dynamics.

The connectivity-performance relationships depicted in FIGS. 6C and 6D further indicate that first-order causal connectivity provides a superior predictor of performance outcomes relative to functional connectivity derived from spontaneous activity alone. Regression analyses of 90th-percentile episode durations as a function of functional connectivity yield moderate coefficients of determination, whereas regressions based on first-order causal connectivity values achieve markedly higher coefficients of determination, particularly within the subset of cycles that reach proficiency. The regression clusters corresponding to proficient and non-proficient cycles separate more clearly along the first-order causal connectivity axis than along the functional connectivity axis, demonstrating that directed, stimulus-locked pathways between selected input and output units are more informative of learning capability than undirected correlation measures. In proficient cycles, first-order causal connectivity values between designated input and output units exhibit especially strong correlation with performance, thereby validating the use of these metrics to guide neural configuration selection.

The feature-correlation analysis summarized in FIG. 6E additionally shows that connectivity features associated with the selected input and output units, such as first-order causal connectivity magnitude and the capacity of output units to evoke multi-order responses and network-wide bursts, display stronger positive correlations with performance than connectivity features associated with other units. Features such as burst probability or functional coupling among non-input/output units exhibit weaker or non-significant correlations, indicating that not all aspects of network connectivity contribute equally to successful learning. The prominence of first-order causal connectivity and output-driven multi-order recruitment in the feature correlation profile highlights that effective neural interfaces preferentially engage neurons that both receive well-structured input from encoding units and can robustly influence broader network dynamics when generating control signals.

Moreover, the comparative performance between cycled adaptive and continuous adaptive paradigms indicates that sustained exposure to performance-contingent training pulses in a continuous adaptive regime enhances the probability of achieving and maintaining proficiency over extended time periods. While cycled adaptive experiments already improve success rates relative to null and random conditions, continuous adaptive experiments exhibit higher fractions of cycles exceeding the proficiency threshold and more frequent segments of high performance. This suggests that repeatedly interrupting adaptive training with null or random cycles can disrupt favorable network states, whereas maintaining an adaptive regime allows the value-estimation and pattern-selection mechanisms to more effectively exploit and stabilize dynamical regimes that support proficient control.

Collectively, the results represented in FIGS. 6A-6E demonstrate that the disclosed characterization and configuration procedures, which compute first-order causal connectivity values and multi-order connectivity values and use these metrics to select input, output, and training neural units, provide a predictive basis for identifying neural configurations that are more likely to support goal-directed learning. By establishing statistically significant relationships between connectivity metrics and high-percentile performance, and by showing that continuous adaptive training further amplifies these advantages, these embodiments confirm that the system is configured not merely to observe learning behavior, but to prospectively optimize neural interfacing parameters in a manner that enhances learning capacity of the biological neural network.

Accordingly, the present disclosure provides an integrated framework that couples cortical organoids with high-density electrophysiology and an adaptive training architecture to induce goal-directed learning in a simulated dynamical control task. The disclosed systems and methods characterize causal connectivity in biological neural networks, designate input, output, and training neural units based on quantified connectivity metrics, and implement closed-loop interaction with a cartpole environment using rate-coded encoding, decoding, and performance-dependent training stimulation. By leveraging first-order and multi-order stimulus-evoked responses to guide neural configuration and by selectively delivering value-optimized training pulses only when task performance declines, the disclosed technology enables sustained improvements in control proficiency that exceed both random stimulation and null conditions, while revealing connectivity features that predict learning capability. In various embodiments, the BrainDance platform operationalizes this framework in a reproducible, extensible manner, thereby enabling practitioners to systematically explore biological learning rules, design hybrid bio-electronic computing systems, and adapt the disclosed principles to other neural preparations, task environments, and training schemes.

### Computer Architecture

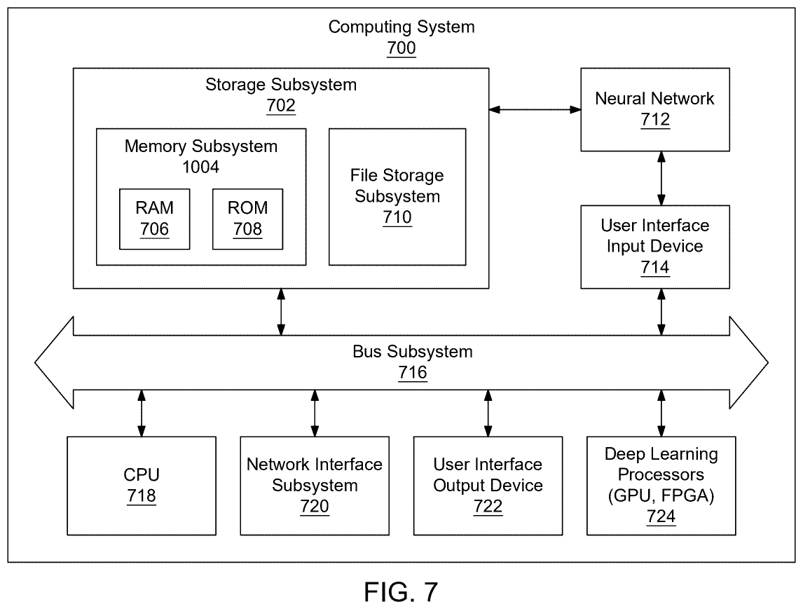

FIG. 7 illustrates a computing system 700 for implementing one or more computational aspects of the present disclosure, including, for example, execution of instructions for characterizing a biological neural network, operating a simulated task environment in closed loop, evaluating task performance, and adaptively selecting and delivering training electrical stimulation patterns. The computing system 700 is provided as one non-limiting example of a suitable computing platform and is not intended to suggest any limitation as to the scope of use or functionality. Regardless of the particular configuration, the computing system 700 is capable of implementing any of the functionality described herein.

The computing system 700 may be implemented using any of a variety of general-purpose or special-purpose computing environments or configurations. Examples of suitable computing systems, environments, and configurations include, without limitation, personal computers, server computer systems, thin clients, thick clients, handheld or laptop devices, multiprocessor systems, microprocessor-based systems, set-top boxes, programmable consumer electronics, network PCs, minicomputer systems, mainframe computer systems, and distributed computing environments that include any of the foregoing systems or devices.

The computing system 700 may be described in the general context of computer system-executable instructions, such as program modules, being executed by one or more processors. Program modules may include routines, programs, objects, components, logic, data structures, and the like that perform particular tasks or implement particular abstract data types. The computing system 700 may be practiced in distributed computing environments where tasks are performed by remote processing devices that are linked through a communications network. In a distributed computing environment, program modules may be located in both local and remote computer system storage media including memory storage devices.

As depicted in FIG. 7, the computing system 700 includes a storage subsystem 702, a bus subsystem 716, a central processing unit (CPU) 718, a network interface subsystem 720, and a user interface output device 722. The storage subsystem 702 further includes a memory subsystem 704 and a file storage subsystem 710. The memory subsystem 704 includes random access memory (RAM) 706 and read-only memory (ROM) 708, which together provide system memory for storing program instructions and data that are immediately accessible to the CPU 718 during operation.

The bus subsystem 716 couples the CPU 718, the storage subsystem 702, the network interface subsystem 720, the user interface output device 722, and a user interface input device 714. The bus subsystem 716 represents one or more of any of several types of bus structures, including a memory bus or memory controller, a peripheral bus, an accelerated graphics port, and a processor or local bus using any of a variety of bus architectures. By way of example and not limitation, such architectures may include an Industry Standard Architecture (ISA) bus, a Micro Channel Architecture (MCA) bus, an Enhanced ISA (EISA) bus, a Video Electronics Standards Association (VESA) local bus, a Peripheral Component Interconnect (PCI) bus, a Peripheral Component Interconnect Express (PCIe) bus, and an Advanced Microcontroller Bus Architecture (AMBA) bus.

The computing system 700 typically includes a variety of computer system readable media. Such media may be any available media that is accessible by the computing system 700 and may include both volatile and non-volatile media, and removable and non-removable media. System memory provided by the memory subsystem 704 can include computer system readable media in the form of volatile memory, such as RAM 706 and/or cache memory. The computing system 700 may further include other removable or non-removable, volatile or non-volatile computer system storage media within the file storage subsystem 710. By way of example only, the file storage subsystem 710 may include one or more non-removable, non-volatile storage devices such as magnetic hard drives, solid-state drives, or other mass-storage devices. In other implementations, the file storage subsystem 710 may also support removable storage, such as magnetic disks, optical disks (for example, CD-ROM or DVD-ROM media), or other removable non-volatile media, connected to the bus subsystem 716 through one or more data media interfaces.

The memory subsystem 704 may store at least one program product having a set of program modules configured to carry out the functions described herein, including the operations for characterizing putative neural units, computing connectivity information, operating the simulated dynamical task environment, decoding control signals, evaluating task performance, and adaptively selecting and delivering training electrical stimulation patterns. A program/utility having one or more program modules may be stored in the memory subsystem 704, together with an operating system, one or more application programs, other program modules, and program data. Each of the operating system, application programs, other program modules, and program data, or some combination thereof, may implement a networking environment and the closed-loop control functionality associated with the present disclosure. The program modules generally carry out the functions and methodologies of the embodiments described herein.

Computer readable program instructions for carrying out operations of the present disclosure may be assembler instructions, instruction-set-architecture (ISA) instructions, machine instructions, machine-dependent instructions, microcode, firmware instructions, state-setting data, or either source code or object code written in any combination of one or more programming languages, including object-oriented programming languages such as Smalltalk or C++, and conventional procedural programming languages such as the C programming language or similar languages. The computer readable program instructions may execute entirely on the computing system 700, partly on the computing system 700 and partly on a remote computer, or entirely on a remote computer or server. In the latter scenario, the remote computer may be connected to the computing system 700 through any type of network, including a local area network (LAN) or a wide area network (WAN), or through an external network such as the Internet using an Internet service provider. In some implementations, electronic circuitry such as programmable logic circuitry, field-programmable gate arrays (FPGA), or programmable logic arrays (PLA) may execute the computer readable program instructions by utilizing state information of the instructions to personalize the electronic circuitry to perform aspects of the present disclosure.

Aspects of the present disclosure may be described with reference to flowchart illustrations and/or block diagrams of methods, systems, and computer program products. It will be understood that each block of such flowcharts and/or block diagrams, and combinations of blocks, can be implemented by computer readable program instructions. These computer readable program instructions may be provided to the CPU 718 or to another processor of a general-purpose or special-purpose computing device to produce a machine, such that the instructions executed by the processor implement the functions specified in the flowchart or block diagram blocks.

The computer readable program instructions may also be stored in a computer readable storage medium of the storage subsystem 702 that can direct the computing system 700 or another programmable apparatus to function in a particular manner, such that the storage medium having instructions stored therein comprises an article of manufacture including instructions which implement aspects of the functions described herein. The instructions may also be loaded onto the computing system 700 or another device to cause a series of operational steps to be performed so as to implement processes such as the real-time read, environment-update, and conditional training phases of the closed-loop system.

The user interface input device 714 may include one or more devices such as a keyboard, mouse, touch screen, pointing device, or other human-machine interface configured to receive user commands, configuration parameters, or experimental control inputs relevant to operation of the system described in this specification. The user interface output device 722 may include a display, monitor, graphical user interface, speakers, or other output peripherals configured to present task-performance metrics, connectivity visualizations, or real-time status information to an operator. The network interface subsystem 720 enables the computing system 700 to communicate with external systems, databases, remote servers, or laboratory information systems over wired or wireless communication links, thereby supporting remote data storage, collaborative analysis, or distributed experiment control.

Accordingly, the computing system 700 provides a flexible and scalable platform for implementing the computational operations associated with inducing adaptive learning in biological neural networks cultured in vitro, while remaining compatible with a broad range of hardware and software environments.

### CLAUSES

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

### We Disclose the Following Clauses:

### Clause Set 1:

In some embodiments, characterizing the biological neural network comprises recording spontaneous activity for a characterization period between about 5 minutes and about 30 minutes and identifying putative neural units based on firing rate and spike amplitude metrics calculated using a normalized activity function η=(1+\hat{r})(1+0.1|μamp|).

In some embodiments, delivering electrical stimulation comprises delivering charge-balanced biphasic electrical pulses having amplitudes of approximately 400 μV peak-to-peak and durations of approximately 400 μs per phase.

In some embodiments, computing stimulus-evoked responses comprises calculating (i) first-order causal connectivity values representing probabilities of direct stimulus-evoked spikes within a post-stimulus window of about 10 ms to about 20 ms, and (ii) multi-order causal connectivity values representing network-mediated responses within a window of about 10 ms to about 200 ms.

In some embodiments, the system excludes network-wide burst events from multi-order causal connectivity calculations by detecting bursts when spike counts exceed the median plus three median absolute deviations.

In some embodiments, selecting the at least one input neural unit comprises identifying a putative neural unit that evokes network-wide bursts in less than about 30 percent of stimulation trials.

In some embodiments, selecting the at least one output neural unit comprises selecting a putative neural unit that demonstrates a first-order causal connectivity probability exceeding a predetermined threshold value.

In some embodiments, the plurality of training neural units comprises between about 5 and about 15 neural units selected independently of causal connectivity patterns.

In some embodiments, operating the simulated task comprises interacting with an inverted-pendulum or cartpole system having state variables including at least the pole angle θ and pole angular velocity θ^{⋅}, wherein episode termination occurs when |θ| exceeds approximately 16 degrees.

In some embodiments, encoding the task state comprises applying stimulation frequencies defined by

$${f}_{1} = {a ( - sin ( θ ) + b )}^{n} and$$

$${f}_{2} = {a ( sin ( θ ) + b )}^{n}$$

with a=7 and b=0.15.

In some embodiments, decoding electrical activity comprises computing smoothed firing rates rₜ=αrₜ_{^{−1}}+(1−α)cₜ, where α=0.2 and cₜ is the spike count within the decoding window.

In some embodiments, adaptively selecting training electrical stimulation patterns comprises updating value estimates according to a temporal-difference learning rule with eligibility traces

$$Vi , t + 1 = Vi , t + α ( Rt - Vi , t ) Ei , t$$

with α=0.3 and Ei,t updated by Ei,t=γEi,t−1+Ii,t using γ=0.3.

In some embodiments, the training stimulation patterns comprise sequences of biphasic pulses delivered at 10 Hz for approximately 300 ms with inter-pulse intervals of approximately 5 ms to 10 ms.

In some embodiments, training stimulation is delivered only when a short-term performance metric based on the last 5 episodes falls below a long-term metric based on the last episodes.

### Clause Set 2

In some embodiments, connectivity information comprises calculating first-order causal connectivity values representing probabilities of direct stimulus-evoked action potentials occurring within a first post-stimulus time window following stimulation and calculating multi-order causal connectivity values representing network-mediated responses occurring within a second post-stimulus time window following stimulation that is longer than the first post-stimulus time window.

In some embodiments, adaptively selecting the training electrical stimulation patterns comprises maintaining value estimates for candidate training stimulation patterns, updating the value estimates based on changes in the task performance, and selecting subsequent training stimulation patterns according to the updated value estimates.

In some embodiments, updating the value estimates comprises applying a temporal-difference learning algorithm with eligibility traces, including updating a value estimate Vi for a candidate training stimulation pattern i according to:

$$Vi , t + 1 = Vi , t + α ( Rt - Vi , t ) Ei , t ,$$

where Vi,t represents a value estimate at time t, α represents a learning rate, Rt represents a reward signal based on task performance, and Ei,t represents an eligibility trace that is updated according to:

$$Ei , t = γ Ei , t - 1 + Ii , t ,$$

where γ represents a decay factor and Ii,t indicates whether the candidate training stimulation pattern i was delivered.

In some embodiments, interfacing the biological neural network with the multi-electrode array comprises positioning a cortical organoid at day approximately 25 of development onto a high-density MEA having at least 500 electrodes, such as about 26,400 electrodes with spacing between about 20 μm and about 30 μm.

In some embodiments, characterizing the network comprises forming a response tensor R∈ [symbol] ^{nreps×nstim×nchannels×nframes} by windowing electrophysiological data over a duration of about 200 ms sampled at approximately 20 kHz.

In some embodiments, selecting neural configurations comprises assigning input, output, and training neural roles based on causal connectivity metrics obtained during the stimulation phase.

In some embodiments, performing closed-loop operation comprises applying a force F to the simulated cartpole system in accordance with the decoded firing-rate difference between output neural units.

In some embodiments, determining task performance comprises computing the episode duration until the simulated pole angle |θ| exceeds approximately 16 degrees.

In some embodiments, adaptively selecting training pulses comprises sampling paired-pulse patterns from a weighted distribution derived from value estimates Vi.

In some embodiments, delivering the selected training pulses comprises stimulating training neural units only when the episode has completed and training criteria are met.

1. A system for inducing adaptive learning in a biological neural network cultured in vitro, the system comprising:
a multi-electrode array configured to interface with the biological neural network and comprising a plurality of recording electrodes and a plurality of stimulation electrodes; and
one or more processors and a memory storing instructions that, when executed, cause the system to:
characterize the neural network by delivering electrical stimulation to a plurality of putative neural units and measuring responses;
select, based on the characterization, a neural configuration comprising at least one input neural unit to receive electrical stimulation encoding task information, at least one output neural unit provide electrical activity for decoding control signals, and a plurality of training neural units to receive training stimulation;
operate a simulated task in a closed loop with the biological neural network by iteratively:
encoding one or more task state variables as electrical stimulation received by the at least one input neural unit;
recording electrical activity from the at least one output neural unit in response to the encoded stimulation and decoding the electrical activity into control signals;
updating the simulated task based on the control signals; and
determining task performance;
adaptively select training electrical stimulation patterns based on the task performance; and
deliver the selected training electrical stimulation patterns to the plurality of training neural units.

2. The system of claim 1, wherein adaptive selection of training electrical stimulation patterns is performed by maintaining value estimates for candidate training stimulation patterns, updating the value estimates based on changes in one or more task-performance metrics, and selecting subsequent training stimulation patterns according to the updated value estimates.

3. The system of claim 2, wherein updating the value estimates comprises applying a temporal-difference learning algorithm with eligibility traces, including updating a value estimate Vi for a candidate training stimulation pattern i according to:
$$Vi , t + 1 = Vi , t + α ( Rt - Vi , t ) Ei , t ,$$
where Vi,t represents a value estimate at time t, α represents a learning rate, Rt represents a reward signal based on task performance, and Ei,t represents an eligibility trace that is updated according to:
$$Ei , t = γ Ei , t - 1 + Ii , t ,$$
where γ represents a decay factor and Ii,t indicates whether the candidate training stimulation pattern i was delivered.

4. The system of claim 1, wherein adaptively selecting the training electrical stimulation patterns further comprises delivering a training stimulation pattern only when a short-term task performance metric calculated over a first number of recent task episodes falls below a long-term task performance metric calculated over a second number of recent task episodes that is greater than the first number of recent task episodes.

5. The system of claim 1, wherein computing connectivity information comprises calculating first-order causal connectivity values representing probabilities of direct stimulus-evoked action potentials occurring within a first post-stimulus time window following stimulation, and calculating multi-order causal connectivity values representing network-mediated responses occurring within a second post-stimulus time window following stimulation that is longer than the first post-stimulus time window.

6. The system of claim 5, wherein the first post-stimulus time window has a duration of about 10 milliseconds following a delivered electrical stimulation pulse, and the second post-stimulus time window extends from about 10 milliseconds to about 200 milliseconds following the delivered electrical stimulation pulse.

7. The system of claim 1, wherein selecting the at least one output neural unit comprises selecting a putative neural unit having a higher first-order causal connectivity value from a candidate input neural unit relative to other candidate output neural units.

8. The system of claim 1, the simulated task comprises a simulated dynamical task environment including an unstable dynamical system requiring continuous active control to maintain a system state within defined bounds.

9. The system of claim 1, comprises an unstable dynamical system comprising an inverted pendulum or a cartpole system having a cart movable along a horizontal axis and a pole rotatably attached to the cart, wherein state variables comprise at least a pole angle and a pole angular velocity, and episode termination is determined based on the pole angle exceeding a threshold angle from vertical.

10. The system of claim 1, wherein the biological neural network comprises a cortical organoid derived from pluripotent stem cells, and the multi-electrode array comprises a high-density microelectrode array configured to record from and to stimulate neural units at a surface of the cortical organoid.

11. The system of claim 1, wherein the plurality of training neural units comprises between 5 and 15 training neural units selected from among the putative neural units and distinct from the at least one input neural unit and the at least one output neural unit.

12. The system of claim 1 wherein each training electrical stimulation pattern comprises a sequence of multiple biphasic electrical pulses delivered to one or more of the plurality of training neural units with an inter-pulse interval of about 5 milliseconds and repeated at a repetition frequency of about 10 Hz for a duration of about 300 milliseconds.

13. The system of claim 1, wherein determining the task performance comprises computing a task performance metric based on durations of episodes of the simulated task, and adaptive selection of the training electrical stimulation patterns achieves a higher fraction of episodes exceeding a proficiency threshold than random selection of training electrical stimulation patterns or operation without training electrical stimulation patterns.

14. The system of claim 1, wherein decoding the control signals from the at least one output neural unit comprises recording spike trains from at least two output neural units, computing smoothed firing rates for the at least two output neural units using exponential smoothing of spike counts over time, and generating the control signals based on a difference between the smoothed firing rates.

15. The system of claim 1, wherein computing connectivity information comprises determining, from stimulus-evoked electrical responses, stimulus-locked action-potential occurrences within defined post-stimulation time windows relative to delivered electrical stimulation pulses.

16. The system of claim 1, wherein selecting the at least one input neural unit comprises excluding putative neural units having a burst-evoking probability exceeding a threshold value, wherein the burst-evoking probability is determined based on a total spike count exceeding a median spike count plus three median absolute deviations.

17. The system of claim 1, wherein the adaptive training stimulation is delivered continuously across successive episodes without cycling through null or random stimulation conditions.

18. The system of claim 1, wherein first-order causal connectivity values between selected input neural units and selected output neural units are predictive of a task-performance capability of the biological neural circuit.

19. A method for inducing goal-directed learning in a biological neural network cultured in vitro, the method comprising:
interfacing the biological neural network with a multi-electrode array comprising a plurality of recording electrodes and a plurality of stimulation electrodes;
characterizing the biological neural network by delivering electrical stimulation to a plurality of putative neural units via the plurality of stimulation electrodes and measuring stimulus-evoked responses via the plurality of recording electrodes;
selecting, based on the characterization, a neural configuration comprising at least one input neural unit configured to receive electrical stimulation encoding task information, at least one output neural unit configured to provide electrical activity for decoding control signals, and a plurality of training neural units configured to receive training stimulation;
operating a simulated task in a closed loop with the biological neural network by iteratively:
encoding one or more task state variables as electrical stimulation delivered to the at least one input neural unit;
recording electrical activity from the at least one output neural unit in response to the encoded stimulation and decoding the recorded electrical activity into control signals;
updating the simulated task based on the control signals; and
determining task performance;
adaptively selecting training electrical stimulation patterns based on the task performance; and
delivering the selected training electrical stimulation patterns to the plurality of training neural units.

20. A non-transitory computer-readable storage medium storing instructions that, when executed by at least one processor coupled to a multi-electrode array interfacing with a biological neural network cultured in vitro, cause the at least one processor to perform operations comprising:
interfacing the biological neural network with a multi-electrode array comprising a plurality of recording electrodes and a plurality of stimulation electrodes;
characterizing the biological neural network by delivering electrical stimulation to a plurality of putative neural units via the plurality of stimulation electrodes and measuring stimulus-evoked responses via the plurality of recording electrodes;
selecting, based on the characterization, a neural configuration comprising at least one input neural unit configured to receive electrical stimulation encoding task information, at least one output neural unit configured to provide electrical activity for decoding control signals, and a plurality of training neural units configured to receive training stimulation;
operating a simulated task in a closed loop with the biological neural network by iteratively:
encoding one or more task state variables as electrical stimulation delivered to the at least one input neural unit;
recording electrical activity from the at least one output neural unit in response to the encoded stimulation and decoding the recorded electrical activity into control signals;
updating the simulated task based on the control signals; and
determining task performance;
adaptively selecting training electrical stimulation patterns based on the task performance; and
delivering the selected training electrical stimulation patterns to the plurality of training neural units.
